(19) United States
(12) Patent Application Publication
(10) Pub. No.: US 2003/0233082 A1
(43) Pub. Date: Dec. 18, 2003

(54) Highly flexible and low deformation fastening device

(75) Inventors: Mark J. Kline, Okeano, OH (US); Miguel Alvaro Robles, Wyoming, OH (US); George Christopher Dobrin, Mason, OH (US); David J. K. Goulait, West Chester, OH (US); James W. Dyess, Summerfield, NC (US); David Porter Welch, West Chester, OH (US); Tracey Elaine Beckman, Milford, OH (US); Jeromy T. Raycheck, Lebanon, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/171,249

(22) Filed: Jun. 13, 2002

(51) Int. Cl. A61F 13/15; A61F 13/20

(52) U.S. Cl. 604/386

(57) ABSTRACT

A strong, easy to use high flexibility, low deformation in plane engagement fastening device suitable for use with articles. The fastening device provides a preferred combination of fastenability, flexibility, load bearing, and minimal deformation. The in plane engagement fastening device simplifies and facilitates proper fastener alignment during the fastening process. The fastening device may a projectile and a receptacle. The projectile is passed into or through the receptacle to engage the fastening device.

### FIELD OF THE INVENTION

The present invention relates to an improved fastening device for absorbent articles such as diapers, training pants and incontinence pads. More particularly, the present invention relates to improved fasteners for joining one portion of a disposable absorbent article to another portion of the article with an improved combination of fit and flexibility for improved comfort with in-plane engagement fastening devices.

### BACKGROUND OF THE INVENTION

Many different types of refastenable fastening devices are known, including ties, pins, hook and loop systems, hook and eye systems, buttons, snaps, interlocking shapes, buckles, adhesive tapes, cohesive surfaces, zippers, and other connectors. Such fasteners have been used on a variety of products, both durable and disposable. Typical uses include clothing, diapers, packages, feminine hygiene products, footwear, and general attachment needs.

Some fastening devices, such as adhesive tapes and hook and loop systems require aligning an engaging surface with a landing surface. While this can result in an effective closure, it often results in misapplication and/or poor alignment of the elements being connected. With an adhesive fastening device, improperly fastening the device may render the entire product unusable. For example, in diaper applications, repositioning a tape tab that has been fastened improperly may result in tearing the outer cover of the diaper and/or a reduction in the adhesion performance of the tape tab adhesive. In order to help prevent such problems, these types of fasteners often require inefficient designs such as extra material usage, which can add to the cost of the products and reduce the flexibility of the fastening device.

Other systems such as buttons, snaps, ties and hooks and eyes, are limited in that they only connect discrete points. Fastening at a discrete point allows material around the fastener to rotate about the discrete points. If more than a single point is connected, these systems generally require more than one fastening device per closure to span the attached area and limit retention. Multiple connections can be cumbersome and may result in gapping between the discrete fastening device components, particularly if the connection is under stress. These systems also require alignment of each fastening device to create the connections desired. Multiple connection fasteners are also typically stiff and as a result, may be uncomfortable to wear.

Other fasteners have no provisions allowing for adjustable fit or alignment during and/or after the fastening process. Poor fastener alignment on a diaper can lead to poor fit, leaks, and undesirable wearer skin marking. There continues to be a need to improve fastening devices, especially for use with disposable products like diapers for improved fit and flexibility. Improved fit can improve the article's performance and a flexible fastener can provide better comfort to the wearer.

Further, deformed fastening devices may bow and deform in a fashion that may allow disengagement or detract from the desired smooth clean lines of a properly fitting and quality article. Fastening device deformation may also contribute to undesirable skin marking on a wearer.

Therefore, it would be advantageous to provide an improved refastenable fastening device suitable for many uses, including disposable absorbent articles, which allows easy connection and an alignment. It would also be advantageous to provide a refastenable fastening device which may adjust, align, and/or conform to the wearer's contours when attached. To this end, it would be desirable to provide a fastening device that readily conforms to different shapes for improved fit when in use, and minimizing skin marking when used on a product to be worn close to the skin. Further, it would be advantageous to provide an absorbent article having a fastening device which provides improved fit and flexibility to the wearer when they move.

### SUMMARY OF THE INVENTION

The present invention is directed to an improved article fastening device. The present invention provides an in plane engagement (IPE) fastening device that provides a preferred combination of fastenability, flexibility, load bearing, and minimal deformation. The in plane engagement fastening device simplifies and facilitates proper fastener alignment during the fastening process. The in plane engagement fastening device may be suitable for use with any article. Suitable articles include disposable absorbent articles such as diapers, catamenial pads, bibs, body wraps, packages, and the like. Other suitable articles include clothing such as a baby one piece outfit, preferably with the fastening device in the outfit's crotch region. The fastening device can also be used on reclosable packages, cartons, bags and other containers.

Modifications in the size, shape, and strength of the in plane engagement fastening device can make it suitable for more high load bearing applications such as seat belts, straps, building materials, etc. Accordingly, the following examples of uses for the fastening device should not be considered to limit the scope of the present invention.

In one embodiment, the article to be fastened includes a fastening device, a first region, a second region opposed to the first region, a tensile load bearing plane (xy-plane) and at least two deflection planes (xz and yz-planes). The fastening device includes a first fastening member and a second fastening member attached to the article and may join at least a portion of the first region with at least a portion of the second region.

In one embodiment, the present invention includes an in plane engagement fastening device comprising a first fastening member, a second fastening member, a tensile load bearing plane, and at least two deflection planes. When the first fastening member and the second fastening member are fastened, the fastening device has a body conformity greater than 200 percent (%) deflection per kilogram force (kgf).

In one embodiment, the fastening device is designed to be flexible without disengaging. Preferably, the fastening device will remain fastened under typical loading and without significant fastening device deformation in the xy-plane. Flexibility in the xz and yz-planes allows the fastening device to bend or deflect out of the way of the wearer's movement. The combination of load and flexibility is achieved by controlling the material properties and part dimensions in the cross-sectional planes (xz-plane, xy-plane, and yz plane). The low fastening device deformation may help maintain the fastener in a fastened configuration and improve the aesthetics of the fastening device on the wearer.

All documents cited are, in relevant part, incorporated herein by reference; the citation of any document is not to be construed as an admission that it is prior art with respect to the present invention.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like numerical designations are used to designate substantially identical elements, and in which:

FIG. 1 is a perspective view of the present invention on an absorbent article;

FIG. 2A is a plan view of a fastener of the present invention engaged;

FIG. 2B is an end view of the embodiment of the fastening device shown in FIG. 2A;

FIG. 3 is a perspective view of the present invention in a fastened configuration;

FIG. 4 is a perspective view of a beam under load in the z-direction;

FIG. 5 is a perspective view of a simplified slot member under load in the x-direction;

FIG. 6 is a plan view of a fastening device on an otherwise conventional absorbent article in its flat-out, uncontracted state with the body-facing surface of the absorbent article facing the viewer;

FIG. 7 is a perspective view of a belted absorbent article;

FIG. 8 is a perspective view of an embodiment of the present invention on a piece of baby clothing;

FIG. 9 is a plan view of a tab member;

FIG. 10A is a plan view of a tab member;

FIG. 10B is an end view of the embodiment of the fastening device shown in FIG. 10A;

FIG. 11 is a plan view of a tab member;

FIG. 12 is a plan view of a slot member and a tab member with longitudinal overhang;

FIG. 13 is a perspective view of a tab member;

FIGS. 14A-C are a perspective view of a tab member with a tab stiffening engagement portion;

FIGS. 15A-B are a perspective view of a slot member;

FIG. 16 is a plan view of a slot member;

FIG. 17 is a plan view of a slot member;

FIG. 18 is a perspective view of a slot member;

FIG. 19 is a perspective view of a housing slot member;

FIGS. 20A-D are a perspective view of alternate rod and socket in plane engagement fastening devices;

FIGS. 21A-B are a perspective view of alternate rod and socket in plane engagement fastening devices;

FIG. 22 is a perspective view of an combination rod and socket, and tab member and slot member in plane engagement fastening device;

FIG. 23A is a plan view of a body conformity test fixture before a load is applied;

FIG. 23B is a plan view of the body conformity test fixture after a load is applied;

FIG. 23C is a plan view of a body conformity test sample;

FIG. 24A is a perspective view of tensile buckling;

FIG. 24B is a plan view of slot deflection;

FIG. 25 is a plan view of a relative deformation test fixture;

FIG. 26A is a plan view of a slot member test sample of the fastening device of the present invention;

FIG. 26B is a cross sectional view of the embodiment of the slot member test sample shown in FIG. 26A;

FIG. 27A is a plan view of a tab member test sample of the fastening device of the present invention;

FIG. 27B is a cross sectional view of the embodiment of the tab member test sample shown in FIG. 27A;

FIG. 28A is a plan view of a tab member test sample of the fastening device of the present invention;

FIG. 28B is a cross sectional view of the embodiment of the tab member test sample shown in FIG. 28A.

### DETAILED DESCRIPTION OF THE INVENTION

While this specification concludes with claims particularly pointing out and distinctly claiming that which is regarded as the invention, the invention can be more readily understood through the following detailed description and drawings.

The present invention provides a flexible yet secure fastening device. Various aspects of the invention are herein described in terms of an absorbent article such as a diaper. However, it is readily apparent that the present invention may also be used to fasten other articles such as disposable absorbent training pants, incontinence briefs, incontinence undergarments, absorbent inserts, diaper holders and liners, feminine hygiene garments, bibs and any other article wherein a fastening device with the characteristics herein disclosed is desired.

Definitions

The terms used herein have the following meanings:

“Absorbent article” refers to devices that absorb and contain liquid. Absorbent articles are generally placed against or in proximity to the body of the wearer to absorb and contain the various exudates discharged from the body. Two examples include diapers and feminine panty liners.

“Disposable” is used herein to describe articles that are generally not intended to be laundered or otherwise restored or reused. For example, they are intended to be discarded after a single use and, preferably, to be recycled, composted or otherwise discarded in an environmentally compatible manner.

“Disposed” is used to mean that an element(s) is formed (joined and positioned) in a particular place or position as a unitary structure with other elements or as a separate element joined to another element.

“Diaper” refers to an absorbent article generally worn by infants and incontinent persons about the lower torso.

“Impermeable” i.e. “liquid impervious” generally refers to articles and/or elements that are not penetrative by fluid through the entire z-directional thickness of the article under pressure of 0.14 lb/in² (0.965 kilopascal) or less. Preferably, the impermeable article or element is not penetrated by fluid under pressures of 0.5 lb/in² (3.447 kilopascal) or less. More preferably, the impermeable article or element is not penetrated by fluid under pressures of 1.0 lb/in² (6.89 kilopascal) or less.

“Joined” encompasses configurations whereby an element is directly secured to another element by affixing the element directly to the other element, and configurations whereby an element is secured indirectly or directly to another element by affixing the element to intermediate member(s), which in turn are affixed to the other element.

“Directly joined” refers to elements which are joined to each other without any intermediate elements joined there between, except for the means joining the elements (e.g. the adhesive).

“Indirectly joined” refers to elements joined with each other by means of an element or elements other than the joining means.

“Body conformity” refers to the percent deflection of a fastening device in the fastened configuration per force (kgf) of compressive deflection load of a fastening device. The body conformity of a fastening device may be measured with the fastening device in a fastened configuration with the first fastening member and the second fastening member of the fastening device interlocked. The body conforming value is normalized for fastening device length in accordance with the body conformity test method. Generally, a higher body conformity is more desirable than a lower body conformity capacity.

“Deflection” refers to bending or moving the fastening device with respect to the article or other locations on the fastening device. For example, a sheet of paper is very flexible and can deflect in many directions. Deflection is generally caused by non-tensile loads.

“Deformation” refers to stretching or shrinking the fastening device. Generally, deformation occurs under a tensile load.

“Relative deformation” refers to 1) the percent of fastening device extension in a direction (x-direction) per kg of tensile load in a direction (x-direction). The relative deformation value is normalized for fastening device length in accordance with the relative deformation test method. Generally, a lower relative deformation value is more desirable than a higher relative deformation value.

“Comprise,” “comprising,” and “comprises” are open ended terms that specify the presence of what follows e.g. a component, but does not preclude the presents of other features, elements, steps or components known in the art, or disclosed herein.

“Compression” refers to a generally compressive load applied at an angle defined in the Body Conforming test method.

“Tension” refers to a generally stretching load or force. The specific application of tension is defined in the Relative deformation test method.

“Engagement” refers to the process of connecting a first fastening member with a second fastening member. Engagement for an in plane engagement fastening device begins when at least a portion of the first fastening member occupies the same three dimensional space coordinates as at least a portion of the second fastening member. For example, when a tab fastening member enters a slot fastening member. Engagement for an out of plane engagement fastening device begins when the fastening device starts to have at least a minimal contact between the first fastening member and the second fastening member and a load carrying capacity is created.

“Fastened” refers to when engagement is complete and the in plane engagement fastening device is configured to maintain a connection between the article first portion and the article second portion.

“Alignment” refers to the designed relative position of the first fastening element and the second fastening element in the xy-plane when fastened.

“Alignment Step” refers to the step which results in the initial relative position of the first fastening element and the second fastening element in the xy-plane.

“Fastening System/Device” refers to everything included to align and engage a first region of an article to a second region of an article. These regions may be part of the same or different articles. The fastening device has a first fastening member and a second fastening member that are joined or fastened to connect the first region with the second region. The fastening device is designed to carry a load that would otherwise separate the two regions. The fastening device first and second fastening members may be the male and female members of an interlocking fastening device.

“Female Member” refers to, for interlocking fasteners, the part of the fastener into which at least a portion of the male member is inserted such as a slot, socket, or receptacle. For non-interlocking interlocking fasteners, the female member is the target at which the male member is placed.

“Male Member” refers to, for interlocking fasteners, the part of the fastener which is (or includes the portions which are) inserted into the female member or a portion of it. Male members may include a tab, ball, rod, or projectile. For non-interlocking fasteners, the part which is placed upon the female member to create a connection is the male member.

“Member” refers to all parts of the fastener, including elements, sub-elements, gripping aids, mechanical assist means, etc.

“Elements” refers to the portions or components of the member. “Subelements” refers to portions of the elements which further create the connection desired. For example, a hook-like element may make a primary connection with a loop-like element, but adhesive on the surface of either element is considered a sub-element.

“Retaining Mechanism” refers to the portion of the fastener which results in the maintenance of the connection. It can be on a member, an element, and/or on a subelement.

As used herein, the term “continuous” as it refers to the line of attachment 72 means generally uninterrupted or unbroken. The term “intermittent” as it refers to the line of attachment 72 means broken or discontinuous.

Discussion

The present invention is directed toward the creation of flexible in plane engagement fasteners.

The Coordinate System

The concept and advantages of flexible in plane engagement fastening devices are best explained in the context of a well defined coordinate system. The coordinate system used for the present invention includes x, y, z directions or axes and xy, xz and yz-planes.

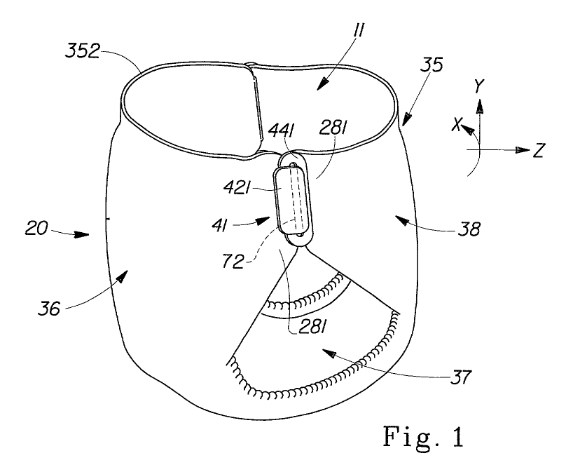

The “x-direction” extends along the surface of the fastening device and/or at least one piece of the article in a direction parallel to the load that the fastening device is designed to carry. Preferably, the load is a tensile load. The x-direction may be called the “lateral” or “transverse” direction. The x-direction is generally orthogonal to both the longitudinal or y-direction and the normal or z-direction at any point on the fastening device and/or article. When the article is a diaper observed as if on a wearer as shown in FIG. 1, the x-direction extends circumferentially in the direction of the load carried by the fastened fastening device. The x-direction may include directions within ±45° of the designed primary load bearing direction. The “primary load bearing direction” is the direction of the tensile load that the fastening device is designed to carry.

The “y-direction” extends along the surface of the fastening device or at least one piece of the article in a direction generally perpendicular to the primary load bearing direction. The y-direction may be called the “longitudinal direction.” The y-direction is generally orthogonal to both the lateral x-direction and the z-direction. When the article is a diaper observed on a wearer as shown in FIG. 1, the y-direction extends vertically along the surface of the fastening device, perpendicular to the direction of the load carried by the fastened fastening device. The y-direction may include directions within about ±45° of the perpendicular to the tensile load that the fastening device is designed to carry and/or within about ±45° of the perpendicular to the z-direction.

The “z-direction” is generally orthogonal to both the x-direction and the y-direction. The z-direction extends out of the surface of the fastening device or at least one piece of the article. The z-direction may be generally perpendicular to the primary load bearing direction. The z-direction may be called the “normal,” or “peel” direction. When the article is a diaper 20 observed on a wearer as shown in FIG. 1, the z-direction extends out of the surface of the fastening device 41, perpendicular to the direction of the load carried by the fastened fastening device 41. The z-direction may include directions within ±45° of the perpendicular to the load that the fastening device is designed to carry and/or 45° of the perpendicular to the y-direction.

Each direction defines an axis about which may be an axis of rotation. For example, the z-axis of rotation is rotation about the z-axis. The rotation used herein will generally follow the right hand rule for positive rotation. All directions will be discussed in a positive orientation when possible since the positive and negative directions of the coordinate system are generally interchangeable as applied herein, except where specifically noted.

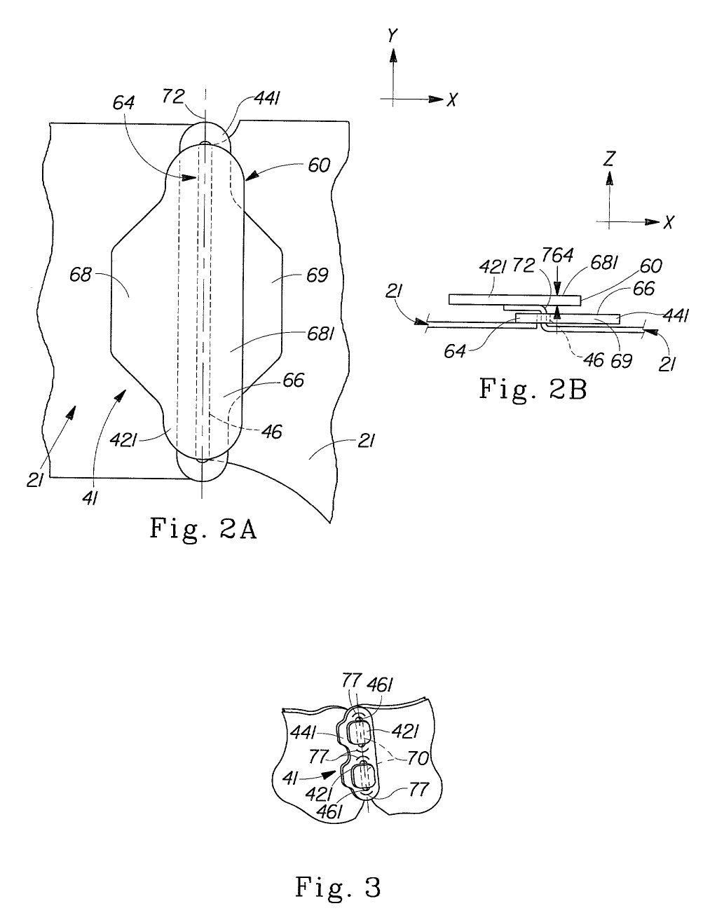

The “xy-plane” i.e. the “tensile load bearing plane” refers to the plane generally congruent with the longitudinal and transverse directions, which generally correspond to the surface of the fastening device. As used herein the xy-plane corresponds to the surface of the fastener as shown in FIG. 2A. A fastened in plane engagement fastening device with a first fastening member and second fastening member may wrinkle/buckle out of the xy-plane as the fastening members distribute loads placed upon them, but the major distribution of stresses is designed to be in the xy-plane. Also, as the overall fastening device conforms to a surface, the plane may form a contoured plane as opposed to a flat plane. For example, the xy-plane may s curve to form a cylindrical- or other curved-surface as shown in FIG. 1. At a specific area of the fastening device 41 in the xy-plane, the z-direction is generally normal to the xy-plane.

The “yz-plane” and “xz-plane” i.e. the “bending or deflection planes” are generally perpendicular to the load bearing plane (xy-plane). The xz-plane extends about the y-direction. The yz-plane extends about the x-direction. Flexibility about these axes into the bending planes may provide an improved in plane engagement fastening device. For example, the in plane engagement fastening device 41 shown in FIG. 1, may conform to the body of the wearer and provide a comfortable fit with minimum skin marking.

The “Primary in-plane direction of engagement” for in plane engagement fasteners refers to the in-plane direction of engagement that includes the greatest displacement (movement) during engagement, excluding any out-of-plane (z) directions. Thus, it is by default either the x-direction or the y-direction. If the x-direction and y-direction displacements are equal during engagement (engagement includes exactly a 45 degree angle motion relative to the primary load bearing direction), then the x-direction is selected as the primary in plane direction of engagement.

In Plane Engagement vs. Out of Plane Engagement

The present invention is directed toward in plane engagement fastening devices. In plane engagement fastening devices include fastening devices such as buckles, hooks & eyes, buttons, tab and slot, interlocking rings/shapes, zippers, many forms of interlocks such as seat-belt buckles, and the like. Non-in plane engagement fasteners are out of plane engagement (OPE) fasteners. Out of plane engagement fasteners include fastening devices such as adhesive or cohesive tapes, hook and loop fasteners, snaps, interlocking shapes/bubbles on the surfaces of two items being connected (e.g. interlocking ridges & groove in a ZIPLOCK® configuration) and the like.

An in plane engagement fastening device is defined as a fastening device that can have substantial motion in the x- and/or y-directions as a first fastening member and a second fastening member are being engaged. This motion is generally in about the same z-plane. Engagement may include limited motions in the z-direction, but these must be supplemented by substantial motions in the x- or y-direction to achieve engagement. An in plane engagement fastening device also allows alignment to continue after engagement begins, thus facilitating the correct fastening and/or positioning of the fastening device. The alignment and engagement steps are part of the fastening process.

In plane engagement fastening devices are preferably independently fastenable. Independently fastenable is defined as wherein the wearer or the caregiver can fasten the device without the use of a mechanical assist means. An example of a mechanical assist means is the slider on a zipper. Such mechanical assist means may be complicated, stiff, expensive, and prone to failure.

An example of an in plane engagement fastening device is a button and a buttonhole. The button moves parallel to the buttonhole in the x-direction (negative x-direction) to approach the buttonhole. The button and/or buttonhole may be rotated so that when engagement begins, the button and/or buttonhole are orthogonal, but the motion remains parallel to the xy-plane overall and no substantive change in the z-direction occurs. Further, engagement is not complete until the button is through the buttonhole, at which time the motion is substantially parallel in the x and y directions.

It may also be desirable that the engagement of the fastening device 41 be achieved during or after fastening without special attention to alignment on the part of a person attempting to fasten the fastening device 41. It is less likely in normal use to fasten the article in an improper configuration when an in plane engagement fastening device is used. For example, a tab and slot fastener has a fastened configuration that is fixed by the tab and the slot used to fasten the article.

Out of plane engagement fastening device fasteners such as tape and hook and loop are very susceptible to operator error when they are fastened, especially when they are fastened on an active wearer that is moving rapidly in random directions. Even where a hook and loop fastener has a defined area for the fasteners to attach, the attachment may be misaligned with only a portion of one fastening element attached to the other fastening element.

In plane engagement fastening device's of the present type are designed such that when fastened they are completely fastened and fastened in the configuration intended by the product designers. Thus, there is less likelihood of fastener misalignment or inadequate fastening. It may also be desirable that the fastening device 41 be capable of adjusting alignment as the wearer moves to maintain proper fit and improve the performance of the article. For instance, for a diaper 20, the article performance improvement may include improving the feces containment capability.

The in plane engagement fastening device may join at least one first fastening member 42 with at least one second fastening member 44 along a continuous line of attachment 72 as shown in FIG. 1, FIG. 2A and FIG. 2B.

FIG. 1 is an example of an in plane engagement fastening device 41. FIG. 1 includes a first fastening member 42 and a second fastening member 44. The two fastening members are fastened along a line of attachment 72. The line of attachment 72 may be formed by at a single point, multiple discrete points, a line, multiple discrete lines, etc. The line of attachment 72 follows a path of connection upon which at least a portion of the load being carried by the first fastening member 42 and second fastening member 44 is carried. The line of attachment 72 may be the actual connected points between the two fastening members, starting at the first y-location being connected by the in plane engagement fastening device and continuing to the last location being connected. The line of attachment 72 may be orthogonal to, at an angle other than 90 degrees to (non-orthogonal), curved, or follow any path relative to the primary direction of load bearing. The line of attachment 72 may extend between multiple tab members in one fastening device, or between fastening devices where multiple fastening devices are used on an article. One example of an extended line of attachment 72 is shown in FIG. 3. The fastening device 41 includes a slot member 441 with two slots 461 and two tab members 421. The slot member also includes multiple slot stiffening members 77. The in plane engagement fastening device may be hermaphroditic in that the male member includes female elements or vice versa.

FIG. 1 shows the fastening device being used on a diaper 20. The diaper 20 includes a first waist region 36, a second waist region 38, a crotch region 37, side panel(s) 281, an article waist 35 and a waist circumference 352.

FIG. 2A and FIG. 2B are more detailed view of an in plane engagement fastening device 41 comprising a tab member 421 and slot member 441 that may be used on any article 21. FIG. 2B is a side end view of the fastening device in FIG. 2A. The line of attachment 72 is shown between the two fastening members. The first fastening member is a male fastening member, and more specifically a tab member 421. The tab member 421 shown includes a proximal edge 60, a tab retaining element 681, and an optional tab grip portion 68. The tab member 421 may also include a multi-plane hinge 727, and a tab thickness 764 in the z-direction. The second fastening member is a female fastening member, and more specifically a slot member 441. The slot member 441 shown includes a slot 46, an inboard portion 64, and an optional slot grip portion 69.

Non-in plane engagement fastening devices are referred to herein as out of plane engagement fastening devices. An out of plane engagement fastening device requires orthogonal motion out of the xy-plane to engage the fastening device 40. An out of plane engagement fastening device is defined as a fastening device that requires the user to align the engaging parts generally in the xy-plane but apart in the z-direction. An out of plane engagement fastening device also requires motion in the z direction (orthogonal to the plane) to engage the fastening device. For example, a tape tab fastener is aligned and brought together in the x-y plane but apart in the z-plane. The separation in the z-plane is then reduced until the fastener is engaged. An out of plane engagement fastening device also does not allow alignment of the fasteners to continue once engagement begins. With a hook & loop or a tape fastener, once the first contact has been made between the first fastening member and the second fastening member (engagement), the alignment, good or bad, of the fastening device is defined and cannot be changed without disengaging the fastening device.

Load and Flexibility Theory Using Beam Analysis

The present invention allows for softer, more flexible in plane engagement fastening is device's than have historically been provided. While flexible, these in plane engagement fastening devices have relatively high load carrying capacity and functionality. Target ranges for flexibility and load bearing are herein disclosed.

Through the analysis of in plane engagement fastening devices and the use of beam bending theories, it is possible to change the bending stiffness and maintain (or improve) the load carrying capacity of a fastening device. This is made possible by careful design of the fastening device and/or paying close attention to where stresses build within the fastening device under load. The fastening device may then be stiffened to carry in plane loads in specific desired locations without a significant increase in the out of plane stiffness of the overall fastening device. The means for providing both flexibility and load bearing capacity involves optimizing cross section designs and materials. The desired characteristics may be achieved by varying the material type or modulus of elasticity (modulus) within the fastening device, varying the fastening device geometry locally, and/or subjecting the finished design to a treatment to locally alter physical properties.

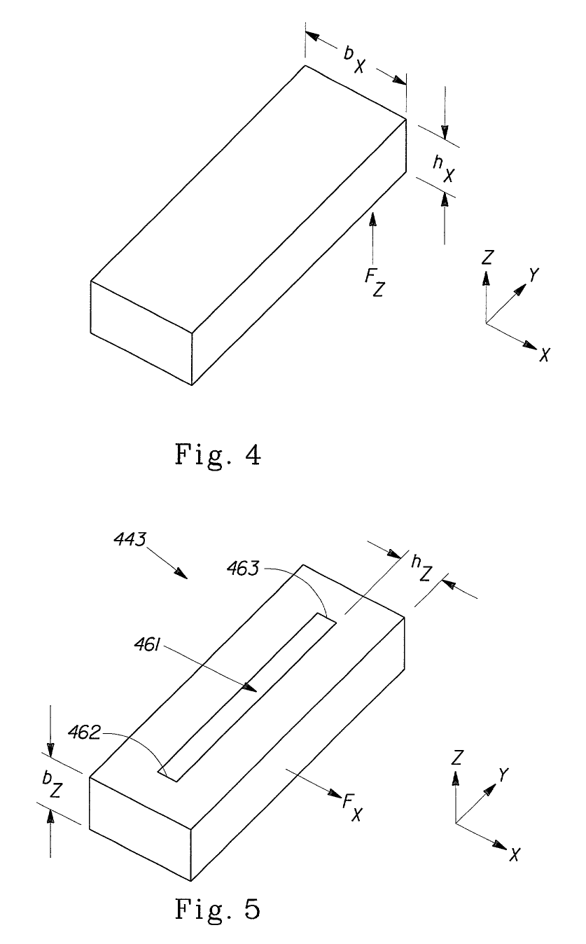

Beam analysis shows that beam bending resistance under a load is proportional to material modulus and the value of b*h³, where b is base of the beam and h is the height of the beam. As shown in FIG. 4, an end load Fz in the z-direction on a beam 18 creates a bending motion about the x-axis. A designator (x) is used to indicate the axis of rotation for the base b and height h measurements. The beam 18 bending analysis of flexibility has the base b(x) extending in the x-direction and the height h(x) extending in the z-direction. The force induces an axis of rotation about the x-axis.

FIG. 5 shows a simplified slot portion 443 of a fastening device with a slot 461. When the simplified slot portion 443 is fastened, there is a distributed force Fx in the x-direction. This causes a bending moment about the z-direction at a first slot end 462 and a second slot end 463. Resistance to the bending force about the z-direction is calculated using the material modulus of elasticity (E) of the material, base b(z) and height h(z). Under this loading it is desirable that there be less flexibility about the z-axis since such flexibility can lead to unsightly fastener deformation and possible fastener disengagement through a deformed slot 461. Unfortunately, the height h(x) being minimized in FIG. 4 for flexibility is the same dimension as the base b(z) which helps reduce the slot 461 deformation when increased. If height h(x) is decreased then base b(z) is decreased and slot deformation may become more pronounced. Previous design challenges with in plane engagement fastening device fasteners include being able to maintain the slot formation and load capacity while providing a flexible and comfortable product for the user. The essential principles herein disclosed can be applied to many shapes and materials to create structures which have high load bearing capability in the x-direction with high z-axis of rotation stiffness (low deformation), yet are very conformable to the body with low stiffness about the y-axis of rotation and/or about the x-axis of rotation.

The fastening devices herein disclosed preferably have a tensile load capacity in at least two perpendicular directions of at least about 100 grams, preferably at least about 500 grams and more preferably at least about 1000 grams. Preferably, the two perpendicular directions are at least one x-direction (e.g. positive or negative x-direction) and at least one y-direction (e.g. positive or negative y-direction).

### ARTICLE EXAMPLES

The fastening device herein disclosed may be used on numerous articles including diapers, clothing, packaging, feminine hygiene products, body wraps, footwear, and the like.

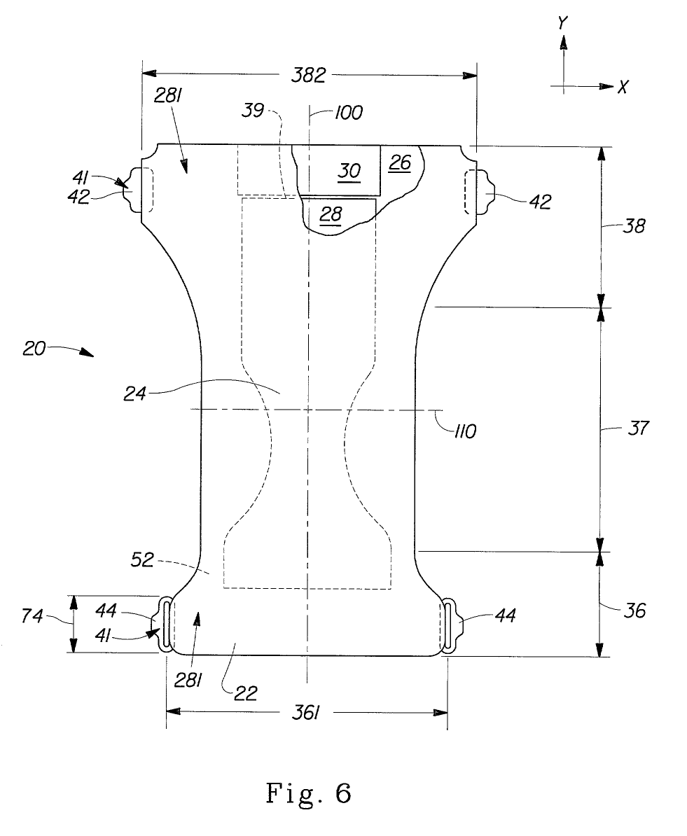

FIG. 6 is a plan view of in plane engagement fastening device 41 attached to a diaper 20 in its flat out, uncontracted state (i.e., without elastic induced contraction). Portions of the structure are cut away to more clearly show the underlying structure of the diaper 20. The portion of the diaper 20 that contacts a wearer is facing the viewer. The diaper 20 has a longitudinal axis 100 and a transverse axis 110. One end portion of the diaper 20 is configured as a first waist region 36. The opposite end portion is configured as a second waist region 38. An intermediate portion of the diaper 20 is configured as a crotch region 37, which extends longitudinally between the first and second waist regions 36 and 38. The crotch region 37 is that portion of the diaper 20 which, when the diaper 20 is worn, is at least partially positioned between the wearer's legs. The waist regions 36 and 38 generally comprise those portions of the diaper 20 which, when worn about a wearer's waist, encircle the waist of the wearer. The fastening device 41 includes a first fastening member 42 and a second fastening member 44 which, on a diaper or similar article, are designed to join the first waist region 36 and the second waist region 38. The waist regions 36 and 38 may include elastic elements such that they gather about the waist of the wearer to provide improved fit and containment. The waist regions 36 and 38 may include side panels 281. The side panels 281 may be elastic and/or extensible.

As shown in FIG. 6, the chassis 22 of the diaper 20 comprises the main body of the diaper 20. The chassis 22 comprises an outer covering including a liquid pervious topsheet 24 and/or a liquid impervious backsheet 26 and at least a portion of an absorbent core 28 encased between the topsheet 24 and the backsheet 26. While the topsheet 24, the backsheet 26, and the absorbent core 28 may be assembled in a variety of well-known configurations, preferred diaper configurations are described generally in U.S. Pat. No. 3,860,003 entitled “Contractible Side Portions for Disposable Diaper” issued to Kenneth B. Buell on Jan. 14, 1975; U.S. Pat. No. 5,151,092 entitled “Absorbent Article With Dynamic Elastic Waist Feature Having a Predisposed Resilient Flexural Hinge” issued to Buell on Sep. 9, 1992; and U.S. Pat. No. 5,221,274 entitled “Absorbent Article With Dynamic Elastic Waist Feature Having a Predisposed Resilient Flexural Hinge” issued to Buell on Jun. 22, 1993; and U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; U.S. Pat. No. 5,580,411 entitled “Zero Scrap Method For Manufacturing Side Panels For Absorbent Articles” issued to Nease, et al. on Dec. 3, 1996; and U.S. Pat. No. 6,004,306 entitled “Absorbent Article With Multi-Directional Extensible Side Panels” issued to Robles et al. on Dec. 21, 1999. The topsheet 24 shown in FIG. 6 may be fully or partially elasticized or may be foreshortened to provide a void space between the topsheet 24 and the absorbent core 28.

The diaper 20 may also include any diaper configuration and/or features known in the art. Exemplary features include breathable backsheets, leg cuffs, front and rear ear panels, waist cap features, elastics and the like to provide better fit, containment and aesthetic characteristics. Suitable alternate diaper embodiments include those disclosed in U.S. Pat. No. 3,860,003 entitled “Contractable Side Portions For Disposable Diaper” issued Jan. 14, 1975; U.S. Pat. No. 5,151,092 entitled “Absorbent Article With Dynamic Elastic Waist Feature Having A Predisposed Resilient Flexural Hinge” issued Sep. 29, 1992; U.S. Pat. No. 6,010,491 entitled “Viscous Fluid Bodily Waste Management Article” issued Jan. 4, 2000; U.S. Pat. No. 5,873,870 entitled “Fit And Sustained Fit Of A Diaper Via Chassis And Core Modifications” issued Feb. 23, 1999; U.S. Pat. No. 5,897,545 entitled “Elastomeric Side Panel for Use with Convertible Absorbent Articles” issued Apr. 27, 1999; U.S. Pat. No. 5,904,673 entitled “Absorbent Article With Structural Elastic-Like Film Web Waist Belt” issued May 18, 1999; U.S. Pat. No. 5,931,827 entitled “Disposable Pull On Pant” issued Aug. 3, 1999; U.S. Pat. No. 5,977,430 entitled “Absorbent Article With Macro-Particulate Storage Structure” issued Nov. 2, 1999 and U.S. Pat. No. 6,004,306 entitled “Absorbent Article With Multi-Directional Extensible Side Panels” issued Dec. 21, 1999.

Generally, when an article such as a diaper 20 is fastened and worn as shown in FIG. 1, there are tensile loads in the x-direction around the diaper 20 and normal or peel loads in the z-direction depending on the motion of the wearer. The motion of the wearer also causes rotational loads about the x-axis. The rotational load on the diaper 20 may be created by the sitting and/or bending of the wearer. A normal load in the z-direction may be created by the wearer's leg movement, bending motion, or their pulling on the diaper 20. The fastening device 41 may deflect in and out of various planes as a result of these loads. Tensile loading of the article is generally in the x-direction around the article waist 35 as shown in FIG. 1. The first fastening member 42 and second fastening member 44 in FIG. 1 carry the tensile load in order to maintain the fastening device 41 in a fastened configuration about the wearer's waist.

The diaper 20 in FIG. 6 may include at least a portion that is extensible and more preferably elastomeric. Preferably, a portion of the first waist region 36 and/or the second waist region 38 is extensible and/or elastomeric. The portion, which is extensible and/or elastomeric, may be located about the lateral centerline 100 of the first waist region 36 and/or the second waist region 38. The extensible and/or elastomeric material may be any known in the art. Exemplary elastomeric and/or extensible waist regions are described in U.S. Pat. No. 5,575,783 entitled “Absorbent Article with Dynamic Elastic Feature Comprising Elasticized Hip Panels” issued Nov. 19, 1996; U.S. Pat. No. 5,749,866 entitled “Absorbent Articles With Multiple Zone Structural Elastic-Like Film Web Extensible Waist Feature” issued May 12, 1998. Preferably, a second extensible and/or elastomeric portion is located in the side panel 281 of the first waist region 36 and/or the second waist region 38.

As shown in FIG. 1, the article waist 35 may have a waist circumference 352. The waist circumference 352 may extend (have an extensibility of) at least about 20% of its unloaded original circumference, preferably at least about 75% and more preferably at least about 200% under a load of less than about 2000 grams (g), and preferably less than about 1200 g, and more preferably under a load of less than 500 g. Waist circumference 352 may or may not return to its unloaded original circumference after a load has been applied and removed. The amount that waist circumference 352 is increased after loading and unloading may be referred to as the percent relaxation. The percent relaxation is preferably less than about 100%, more preferably less than about 50%, and most preferably less than about 10% after a load of less than about 2000 grams has been applied and removed. Alternatively, the percent relaxation is preferably less than about 100%, more preferably less than about 50%, and most preferably less than about 10% after a load of less than about 1200 grams has been applied and removed. More preferably, the percent relaxation is preferably less than about 100%, more preferably less than about 50%, and most preferably less than about 10% after a load of less than about 500 grams has been applied and removed.

The fastening device 41 is preferably located in a location that will be comfortable to the wearer. As shown in FIG. 1, a preferred location for the fastening device 41 is near the side of is the wearer on a diaper 20. Alternatively, it may be preferred that the fastening device 41 be located in a rearward location when worn on the wearer. A rearward location is slightly to the rear of the wearer, between the wearer's outermost side of their thigh and the wearer's buttocks. However, the fastener should not be so far to the rear as to make diaper 20 changes difficult if the baby is lying down.

As shown in FIG. 6, the proper fastener location may be defined by a first waist width 362 and a second waist width 382. In this example, the first waist region 36 corresponds to the position of the diaper 20 positioned at the front of the wearer. First waist width 362 is the width of the first waist region 36, up to the line of attachment 72 (FIG. 1) with the second waist region 38 when the fastening device 41 is fastened. Second waist width 382 is the width of the second waist region 38, up to the line of attachment 72 with the first waist region 36 when the fastening device is fastened. The combined first waist width 362 and second waist width 382 combine to create an article waist circumference 352 shown in FIG. 1. Preferably, in an embodiment where the first waist region 36 is placed in the front of a wearer, the second waist width 382 is less than the first waist width 362. Preferably, in this embodiment, the second waist width 382 is less than the first waist width 362 by about 10% or more.

Preferably, the second waist width 382 is between about 30% of the article waist circumference 352 and about 50% of the article waist circumference 352. Preferably, the second waist width is between about 35% of the article waist circumference 352 and about 45% the article waist circumference 352. These preferred ranges apply to the article in a condition in which no external extension force is applied to the waist hoop. These ranges may also apply to an article, such as a diaper 20, when it is worn about a wearer of appropriate weight and waist diameter for which the diaper was designed.

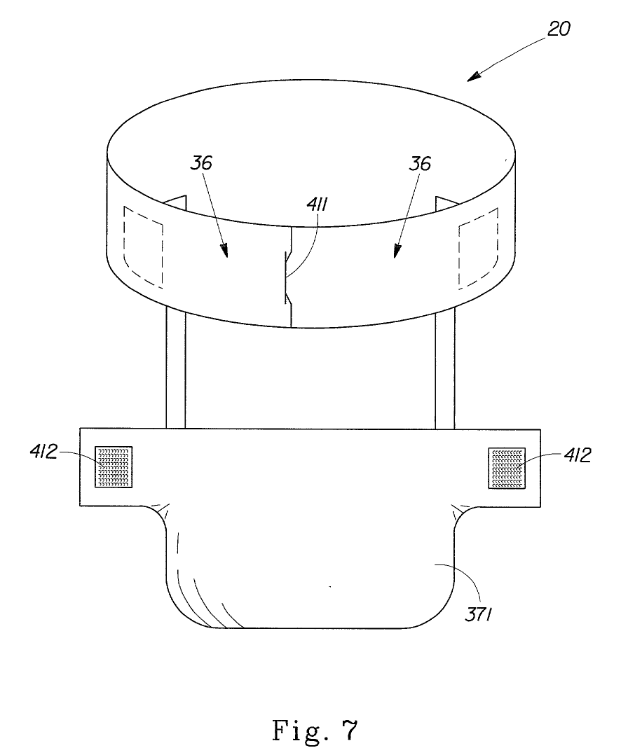

As shown in FIG. 7, a flexible in plane engagement fastening device 411 may be used to join a portion of the first waist region 36 of an article to another portion of the first waist region 36 of the diaper 20. A second fastening device 412 may be an in plane engagement fastening device or an out of plane engagement fastening device. The second fastening device 412 joins the second waist region 38 to the first waist region 36.

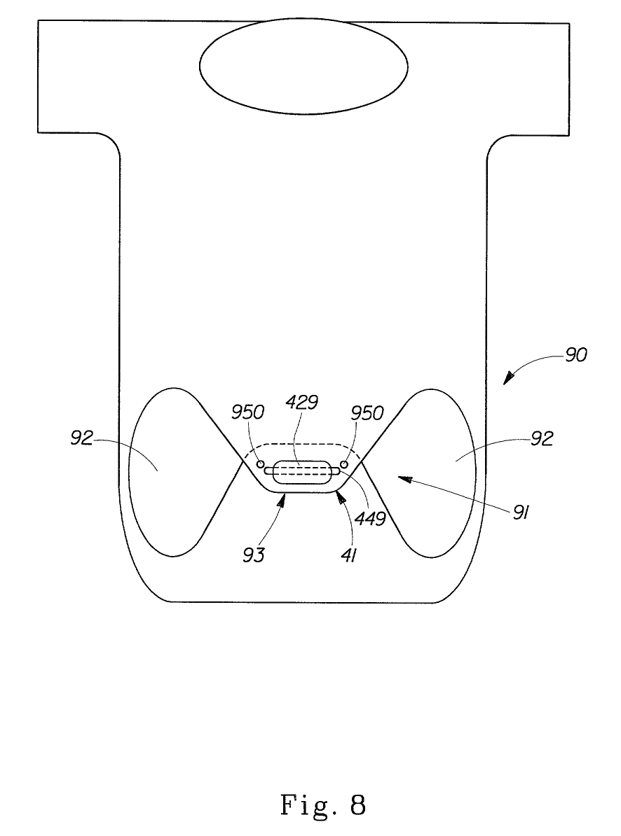

The flexible in plane engagement fastening device disclosed herein may have applications on other articles as well. For example, a flexible in plane engagement fastening device may be used on articles of clothing. One example of this is a one-piece baby outfit 90 as shown in FIG. 8. As shown in FIG. 8, at least one in plane engagement fastening device 41 may be in the crotch region 91 of the one-piece baby outfit 90. The one-piece baby outfit 90 typically has an access point 93 between the two leg openings 92 that are formed when the fastening device 41 is fastened. When unfastened, the fastening device 41 provided an access point 93 for changing under garments such as a diaper. The one-piece 90 may be made out of any material. Common materials include wool, cotton, polyester, combinations thereof, and the like. The in plane engagement fastening device may be any in plane engagement fastening device herein disclosed. As shown in FIG. 8, the fastening device 41 may be a tab member 429 and slot member 449 configuration. The in plane engagement fastening device 41 may optionally be used in combination with other fasteners. For example a snap 950 may be used to secure a portion of the one piece 90 to close the leg openings 92 about the wearer while the in plane engagement fastening device is used to close at least a portion of the remaining access point 93 about the wearer. The in plane engagement fastening device 41 is designed to simplify the alignment and fastening of the one-piece 90 in the proper configuration while having flexibility sufficient to ensure reasonable comfort for the wearer.

Other Fastening Device Capabilities

The in plane engagement fastening device may be configured to fasten the product for disposal in a disposal configuration. A disposal configuration includes any fastened configuration to maintain bodily waste or other refuse within the article after the article is removed from the wearer, at least until the article is subsequently deposited in a refuse container or otherwise removed from the vicinity of the wearer. The in plane engagement fastening device used to fasten the product for disposal may be the same fastener used to fasten the article in the configuration intended for wearing, or it may be a different fastener.

The in plane engagement fastening device may preferably be prefastened before the article is secured in its final location about the wearer. For example, one or more in plane engagement fastening devices may be fastened by the manufacturer prior to being placed in a package such that the end user removes prefastened products from the package. The article may be pulled into place about the wearer without unfastening the in plane engagement fastening devices. Alternatively, the user may prefasten the in plane engagement fastening devices prior to pulling the article into place about the wearer.

Fastening device embodiments may include multiple fastening members to provide adjustment and securement alternatives. For example, more than one tab member 421 or more than one slot member may be place in parallel in the x-direction. Depending upon the use desired, different tab and slot combinations may be used to provide a preferred fit or other fastened configuration.

Specific Alternate In Plane Engagement Fastening Device Embodiments

There are many different in plane engagement fastening devices including buckles, buttons, tabs & slots, zippers, etc. However, there are 2 particularly preferred categories of in plane engagement fastening device's suitable for use as fasteners to be worn in close body contact. The first category is “tab & slot” in plane engagement fastening device and includes any in plane engagement fastening device in which a male member, generally referred to as “a tab member,” includes a “tab” which interlocks with an opening in a female member (generically referred to as a “slot member”). The second category is a “projectile & receptacle” in plane engagement fastening device and includes any in plane engagement fastening device in which a male member includes at least one projectile from a surface which interlocks with at least one matching receptacle of a female member. To make in plane engagement fastening devices both flexible and load bearing, particular attention is paid to both the tab member 421 and the slot member 441 design & materials. The tab members and slot members of tab & slot in plane engagement fastening device's can take various forms, including those examples described below.

Tab Forms

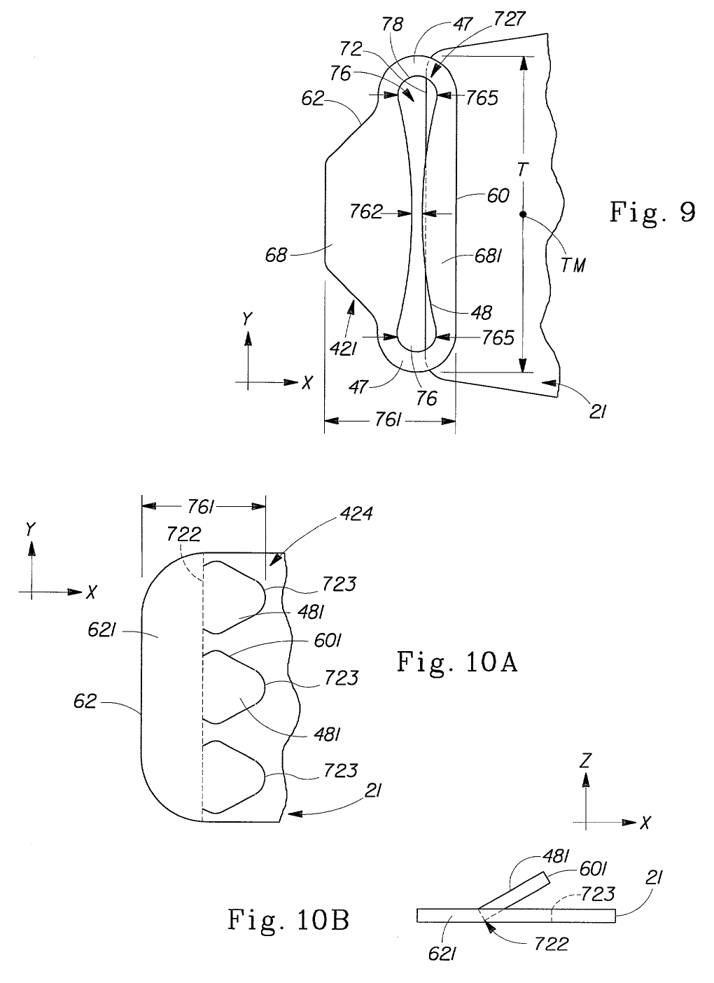

The tab member can take many forms, including the tab shown in FIG. 9. In embodiments where the tab member 421 is used near or against the skin of a wearer, it is preferred that the materials making up the tab member 421 be flexible. The flexibility allows the tab member to conform to the shape of the body and thus, reduces the likelihood that the tab member 421 will irritate or injure the wearer's skin. Further, the material from which the tab member 421 is made can be reinforced and/or weakened at certain locations to help provide the desired flexibility and stiffness to the fastening device. The tab member 421 may be of any size and/or shape and may be made from any suitable material. As shown in FIG. 9, the tab member 421 is preferably an elongated member having a tab length T, a proximal edge 60, a distal edge 62, and a tab retaining element 681 generally adjacent at least a portion of the proximal edge 60. The bisection of the tab length T identifies a tab midpoint TM. The proximal edge 60 may be located over the article 21 laterally inward from the proximal edge 60. The tab member 421 preferably has tab longitudinal ends 47, and a tab stiffening member 78. The tab stiffening member 78 may extend at least partially into the tab load bearing portion 76. The tab stiffening member 78 may be integral with the tab load bearing portion 76. The tab load bearing portion includes a load bearing portion tab end width 765 located near the longitudinal ends 47 and a load bearing portion tab central width 762 located near the tab midpoint TM. The tab member may also include a tab grip portion 68 and a tab width 761. The tab width 761 is measured in the x-direction. The tab member 421 may include a multi-plane hinge 727 as shown in FIG. 2B and FIG. 9. A multi-plane hinge 727 is defined as where at least a portion of the tab member 421 overhangs another portion of the tab member 421 and/or a portion of the article when unfastened and/or fastened. When the fastening device is fastened, the line of attachment 72 generally follows at least a portion of the multi-plane hinge 727.

Tabs with Laterally Overhanging Tab Retaining Elements

The tab member 421 shown in FIG. 9 includes a retaining element 681 configured such that at least a portion of the retaining element 681 laterally overhangs at least a portion of the slot member 441 when the tab member 421 and slot member 441 are in a fastened configuration as shown in FIG. 2A. The tab member in FIG. 2B shows a multi-plane hinge 727. A multi-plane hinged tab member includes a distal edge 60 and/or retaining element 681 that when in a horizontal orientation as shown, overlaps a portion of the article 21, or another portion of the tab member 421.

In one preferred embodiment, when the fastening device is fastened there is at least one location in which at least a portion of the tab member 442 extends in the x-direction over a portion of the slot member 441 as shown in FIG. 2A. As shown in FIG. 2B the tab member 421 may include a line of attachment 72 such that at least a portion of the tab member 421 can pivot about at least the y-axis relative to another portion of the tab member 421 or relative to a portion of the article 21.

Also shown in FIG. 10A and FIG. 10B, the tab member 424 may form a single plane hinge 722. The single plane hinge 722 has no portion of the tab member 424 overhanging another portion of the tab member 424 or a portion of the article when unfastened as shown in FIG. 10A and FIG. 10B. A single plane hinge 722 may be formed by cutting the article 21 along at least one cut line 723 to form at least one proximal edge 601. The cut line 723 may take any path. The cut may go through the article 21 and or a portion of the article 21. The resulting tab member 424 has at least one distal portion 621 and at least one longitudinally overhanging retaining element 481 which overhangs the slot member 44 (FIG. 17) when the in plane engagement fastening device is fastened. The tab width 761 is measured from the distal edge 62 in the x-direction to the proximal edge 601 furthest from the distal edge 60. The singe plane hinge 722 may extend between the overhanging retaining elements 481 as shown.

As shown with two views in FIG. 2A and/or FIG. 2B, the fastening device 41 is fastened by passing the tab member 421 completely through a slot 46 of the slot member 441. Once the tab member 421 has been passed through the slot member 441, the retaining element 681 of the tab member 421 is rotated into a plane generally parallel with the plane of the slot member 441. After rotation, at least a part of the retaining element 681 and a proximal edge 60 are overlapping at least a part of a slot outboard portion 66 of the slot member 441. In this configuration, the retaining element 681 of the tab member 421 will prevent the tab member 421 from slipping back through the slot 46 and disengaging the fastening device 41. A portion of the tab member 421 or the material of the article to which the tab member 421 is joined will extend into and through the slot 46, as shown in FIG. 2B. The overlapping retaining element 681 is designed to resist the tension load in the x-direction, which tends to pull the tab member 421 and the slot member 441 apart. Loads in the z-direction may pull the article tighter about the wearer but will not typically disengage the fastening device 41 without further manual manipulation of the tab member 421 and slot member 441.

Tabs with Longitudinally Overhanging Tab Retaining Elements

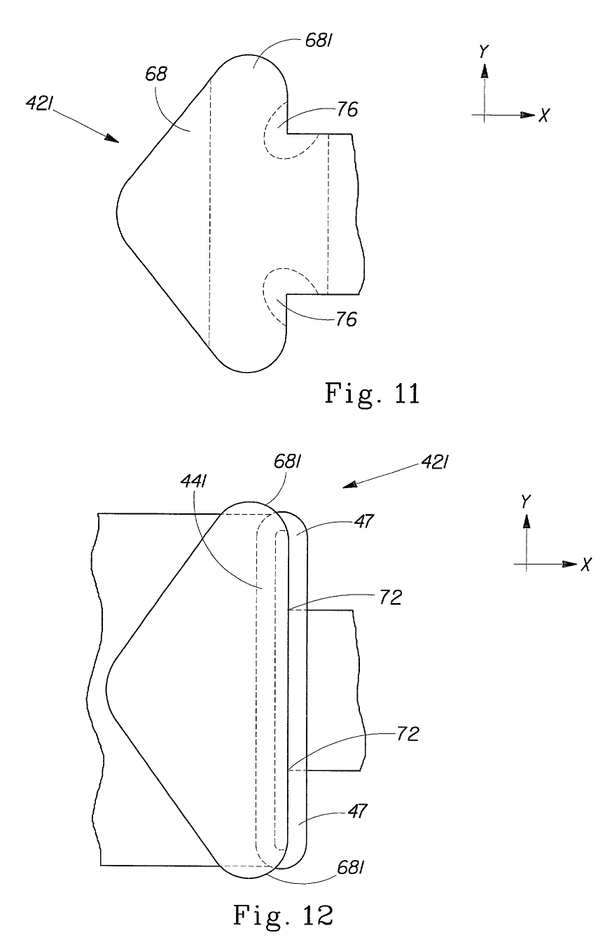

As shown in FIG. 11, in another embodiment, the tab member 421 may include at least one tab retaining element 681. The tab retaining element 681 is configured such that at least a portion of the tab retaining element 681 longitudinally overhangs at least a portion of the slot member 441 (FIG. 12) in the y-direction when the tab member 421 and slot member 441 are in a fastened configuration as shown in FIG. 12. In order to improve the body conformity of the tab member 421, it may be preferred to have at least two tab load bearing portions 76 as shown in FIG. 11. The tab member 421 may also include a grip portion 68.

Preferably, as shown in FIG. 12 the tab retaining element 681 extends over both longitudinal ends 47 of the slot member 441. As shown in FIG. 12, the tab member 441 may laterally and longitudinally overhang at least a portion of the slot member 441. The tab load bearing portions 76 are located along the line of attachment 72.

Tabs with Non-overhanging Tab Retaining Elements

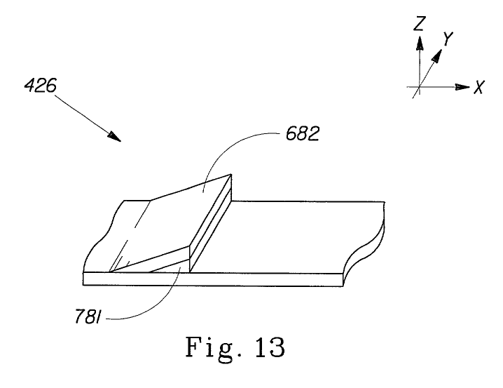

As shown in FIG. 13, the tab member 426 may include a tab retaining element 682 configured such that the tab retaining element 682 does not overhang any portion of the slot member when the tab member and slot member are in a fastened configuration. Instead, the tab retaining element 682 protrudes into the slot of the slot member to resist disengagement.

The tab retaining element 682, shown in FIG. 13 may be movable relative to other portions of the tab member 426 or may be in a fixed position relative to the tab member 426. The tab retaining element 682 preferably includes at least a resilient portion 781 to cause the tab retaining element 682 to return to approximately its original position after a fastening force is applied to it. Engagement of such embodiments may occur via at least a slight elastic deformation of the tab retaining element 682 as the tab member 426 passes into the slot member 444 slot 466 (FIG. 19). Engagement is completed when the tab retaining element 682 returns to approximately its original position and interlocks with at least a portion of the slot member 444. Alternatively, at least a portion of the slot member 444 may be resilient such that it can elastically deform at least a small amount during engagement then return to approximately its original position to fasten with tab retaining element 682. Preferably, both the tab member 426 and slot member 444 have at least a portion which elastically deform at least slightly during engagement. Combinations of overhanging and non-overhanging tab members may be used as a fastening device.

Tab Member Design

As shown in FIG. 9, the tab member 421 may have a tab load bearing portion 76 and a tab grip portion 68. The tab load bearing portion 76 (TLBP) is defined as the portion of the tab member 421 that is located immediately adjacent the slot load bearing portion 67 (FIG. 17) when the tab and slot are fastened. Generally, this corresponds to the area of the first and second fastening member 42 & 44 immediately about the line of attachment 72 (FIG. 1). Preferably, the tab load bearing portion 76 is the portion within about 0 to about 15 mm, of any portion of the tab adjacent the slot load bearing portion 67 when the fastening device 41 is fastened. More preferably, the tab load bearing portion 76 is the portion within about 0 to about 10 mm, of any portion of the tab adjacent the slot load bearing portion 67 when the fastening device 41 is fastened. Most preferably, the tab load bearing portion 76 is the portion within about 0 to about 5 mm, of any portion of the tab adjacent the slot load bearing portion 67 when the fastening device 41 is fastened. The tab grip portion 68 shown in FIG. 9 is defined as the remaining portion of the tab member 421 outside the tab load bearing portion 76.

Although there may be no difference in material properties or structure between the tab load bearing portion 76 and the tab grip portion 68, both the tab load bearing portion 76 and the tab grip portion 68 may have different material properties or structure within their respective portions. Preferably, The tab load bearing portion 76 and tab grip portion 68 have different material properties, and/or structural differences. The material and/or structural differences between the tab load bearing portion 76 and the tab grip portion 68 may include a gradual transition in properties. The tab load bearing portion 76 and the tab grip portion 68 may both have different material properties or structure within their respective portions as well.

For example, as shown in FIG. 9, the tab load bearing portion 76 may include a tab structure with a plastic such as polypropylene of a thickness greater than about 0.25 mm. The plastic may be covered in a relatively light, flexible nonwoven. The nonwoven layer or layers may extend beyond the perimeter of the tab plastic. In such an embodiment, the tab load bearing portion 76 is defined by the portion of the tab member 421 including at least the plastic. The tab grip portion 68 is any other portion of the tab member 421. One example of a tab stiffening member 78 is shown in FIG. 9.

A tab stiffening member 78 is designed to maintain the load bearing capability of the fastening device while still allowing improved flexibility of the overall fastening device 41. The tab stiffening member 78 may be designed to prevent the tab member 421 from folding back onto itself under load and disengaging. The tab stiffening member 78 may be positioned anywhere on the tab member 421. Preferably the tab stiffening member 78 is positioned at least partially adjacent and or between the proximal edge 60 and the distal edge 62. At least a portion of the tab stiffening member 78 may further be located at or near the longitudinal ends 47.

Preferred Materials for Tab Members

The tab member 421 may be of any size and/or shape and may be made from any combination of suitable material. The tab member 421 may be made of materials the same as or different from the slot member including plastics, films, foams, nonwoven webs, woven webs, paper, laminates, metals, fibers, fiber reinforced plastics and the like, or combinations thereof. As with the slot member 44, it is preferred that the materials making up the tab member 421 be flexible. However, the tab member 421 should be stiff enough in the x-direction and/or y-direction so as not to deform and let the tab member 421 disengage under in use fastening forces. The material from which the tab member 421 is made can be reinforced or weakened at certain locations to help provide the desired levels of flexibility and stiffness to the fastening device 41. Preferred plastics for the tab member 421 include polyester, polypropylene, polyethylene, polystyrene, nylon, and the like. Preferred metals include steel, aluminum, copper, tin, brass, combinations thereof, and the like. Suitable fibers may include natural and/or synthetic fibers.

The tab member 421 may be unitary with the article to which it is attached or may be a separate element joined thereto. The tab member 421 may be joined to the article at any location. In order to optimize the body conformity and relative deformation performance of the fastening device, the tab member 421 tab grip portion 68 and tab load bearing portion 76 shown in FIG. 9 may have different materials and properties. The materials and properties may also vary within the tab member 421 grip portion 68 itself and tab load bearing portion 76 itself. Preferably, the tab grip portion 68 is made of material that is thin, with a low modulus of elasticity (flexible). Examples include materials with a thickness less than about 1.0 mm, preferably less than about 0.5 mm, and more preferably less than about 0.25 mm. The material may also have a modulus of less than about 2.0 Gigapascals (Gpa), preferably less than 1.0 Gpa, and more preferably less than 0.5 Gpa. The tab grip portion 68 may have a basis weight less than about 100 gsm, preferably less than about 70 gsm and more preferably less than about 30 gsm.

Examples of preferable tab grip portion 68 materials include nonwovens such as carded, spunbond, meltblown, spunbond-meltblown-spunbond, and the like. The tab grip portion 68 may also be a laminate. The laminates may be of two or more layers of material. Exemplary laminates include nonwoven-nonwoven, nonwoven-film, and the like. Tab grip portion 68 materials may be integral with tab load bearing portion 76. Alternatively, the tab grip portion 68 material may be separate pieces of material attached to the tab load bearing portion 76. Preferably, the entire exterior surface of the tab member 421 is covered in a soft, fuzzy material such as a nonwoven.

Preferably, the tab load bearing portion 76 is made of material that is thin, with a high modulus of elasticity (stiff). The thickness of the tab member 421 is measured in the z-direction as shown in FIG. 2B. Preferred tab load bearing portion 76 material thickness & modulus requirements vary with tab length T and/or the amount of load being carried along the tab member 421. Shorter tab lengths T allow thinner and/or lower modulus materials, while longer tab lengths T require thicker and/or higher modulus materials for ease of use. For relatively short Tab lengths T of less than about 3 cm, relatively thin materials and/or low modulus may be used. The tab load bearing portion 76 preferably has a thickness less than about 0.5 mm and more preferably less than about 0.25 mm. The tab load bearing portion 76 preferably has a modulus greater than about 200 MPa, and more preferably greater than about 500 MPa.

For tab lengths T (FIG. 9) greater than about 3 cm, thicker and/or higher modulus materials are preferred. The tab load bearing portion 76 preferably has a thickness greater than about 0.3 mm and more preferably greater than about 1 mm. The tab load bearing portion 76 preferably has a modulus greater than about 500 MPa, and more preferably greater than about 1000 MPa. For tab load bearing portion 76 materials of extremely high modulus, such as steel, the material thickness may be reduced to less than about 0.2 mm.

The tab grip portion 68 and tab load bearing portion 76 may have a different basis weight (weight/unit area). Preferably, the tab grip portion 68 includes at least one portion having a lower basis weight than at least one portion of the tab load bearing portion 76. Preferably, the ratio of basis weight in the tab grip portion 68 to that in the tab load bearing portion 76 (BW₆₈/BW₇₆) is less than about 1, less than about 0.5, and more preferably less than about 0.25

Tab Load Bearing Portion Parameters

The stiffness within the tab load bearing portion 76 may vary along the x- and/or y-directions. In certain embodiments the tab member 421 and/or slot member 441 may have a constant plan view design wherein the materials and dimensions are the same throughout the member. Preferably, stiffness may be varied by varying plan view area, thickness, basis weight, dimensions, and/or modulus of the tab load bearing portion 76.

In one preferred embodiment shown in FIG. 9, the plan view area of at least a portion of the tab load bearing portion 76 is varied in the xy-plane. This may be achieved by preferably having an load bearing portion tab end width 765 that is wider in the x-direction near at least one tab longitudinal end 47 of the tab member 421 than a load bearing portion tab central width 762 located approximately at about the tab midpoint TM of the tab length T. Preferably, the width ratio of the load bearing portion tab end width 765 to the load bearing portion tab central width 762 is greater than about 1.0, greater than about 1.25, and preferably greater than about 2.

In another preferred embodiment (not shown), the z-direction thickness in the zy-plane of at least a portion of the load bearing portion 67 may be varied along the y-direction. Preferably, the z-direction end thickness near at least one longitudinal end 47 is thicker than a central thickness at about the midpoint TM of the tab length T. Preferably, the thickness ratio of the end thickness to the midpoint thickness in the load bearing portion 67 is greater than about 1.0, greater than about 10, and preferably greater than about 20.

In another preferred embodiment, the modulus of at least a portion of the tab load bearing portion 76 may be varied along the y-direction and/or the x-direction. Preferably, the modulus near at least one tab longitudinal end 47 is higher than the modulus at about the midpoint TM of the tab length T and at the tab load bearing portion 67. Preferably, the modulus ratio of the tab longitudinal end modulus to the midpoint modulus is greater than about 1.0, and preferably greater than about 3, more preferably greater than about 10, and more preferably greater than about 25.

In other embodiments, the tab member 421 may be less stiff about the y-axis by weakening the tab member 421 stiffness in the x-direction. Methods of weakening the tab member include scoring, cutting, thinning, bending, heat treating, chemical treating, and the like.

Tab Dimensions

Preferably, the tab load bearing portion 76 is relatively thin in the z-direction, relatively narrow in the x-direction, and/or relatively long in the y-direction. The preferred z-direction tab thickness 764 (FIG. 2B) may be less than about 5 mm, less than about 3 mm, less than about 1 mm, less than about 0.5 mm. Preferred x-direction tab widths 761 as shown in FIG. 9 are less than about 40 mm, less than about 30 mm, less than about 20 mm, or less than about 15 mm. Preferred y-direction lengths T as shown in FIG. 9 are more than about 20 mm, more than about 30 mm, more than about 50 mm, or more than about 60 mm. Preferably, the tab member 421 and/or the tab load bearing portion 76 have a tab width 761 to tab thickness 764 (FIG. 2B) ratios of more than about 5, more than about 10, more than about 15, more than about 20 or more than about 30. Preferably, the tab member 421 and/or the tab load bearing portion 76 also have a tab length T/thickness 764 ratios of more than about 10, more than about 40, more than about 70, more than about 100. The dimensions and ratios may apply to either the overall tab member 421 and/or the tab load bearing portion 76 within the tab member 421. The tab member 421 stiffness may vary in any direction. The tab member stiffness preferably varies in the y-direction and/or x-direction. In another preferred embodiment, basis weight (weight/unit area) of at least a portion of the tab load bearing portion 76 is varied along the y-direction and/or the x-direction. Preferably, the basis weight near at least one tab longitudinal end 47 is higher than the basis weight in the midpoint TM region of the tab length T. Preferably, the basis weight ratio of the longitudinal end to the midpoint TM region basis weight is greater than about 1, and preferably greater than about 2, and more preferably greater than about 5. The tab member 421 basis weight may vary in any direction. The tab member basis preferably varies in the y-direction and/or x-direction.

Preferably, tab member stiffness varies in the x-direction as well as the y-direction. For example, the basis weight, thickness, and/or modulus of the tab load bearing portion 76 may vary in the x-direction, y-direction, and/or z-direction. The preferred basis weight, thickness, and/or modulus ratios described for longitudinal variations along the length of the tab also apply for lateral variations along the width of the tab.

Tab Stiffening Engagement Portions

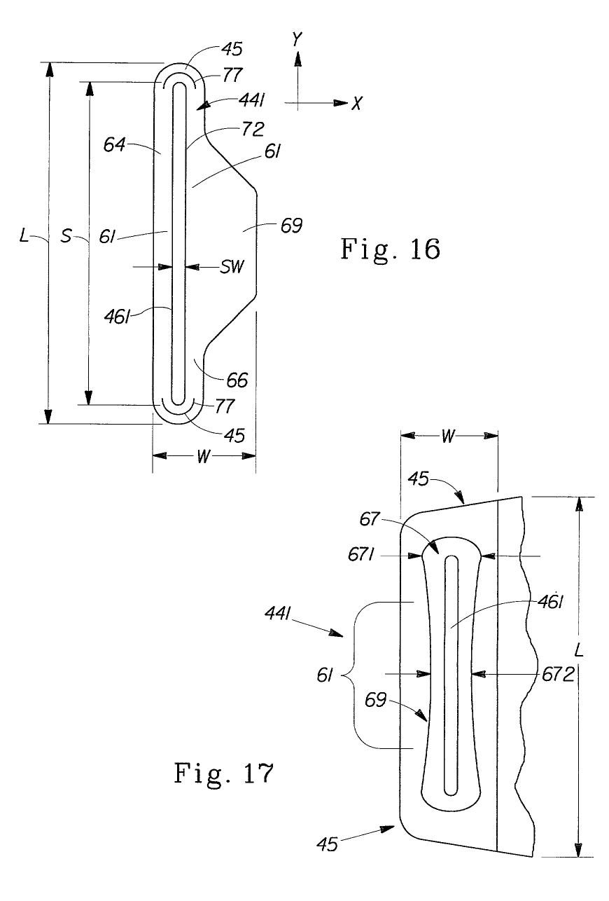

The tab member may include a tab stiffening engagement portion. The stiffening engagement portion provides stiffness in specific location to facilitate the insertion of the tab member into the slot member, preferably with minimal adverse affect on the fastening device 41 body conformity or relative deformation properties. The stiffening engagement portion may be part of the load bearing portion and/or the gripping portion of either the tab member or the slot member. Without the stiffening engagement portion, the tab gripping portion 68 and/or the tab distal edge 62 (FIG. 9) may flex, curl or otherwise resist insertion into the slot 461 (FIG. 16).

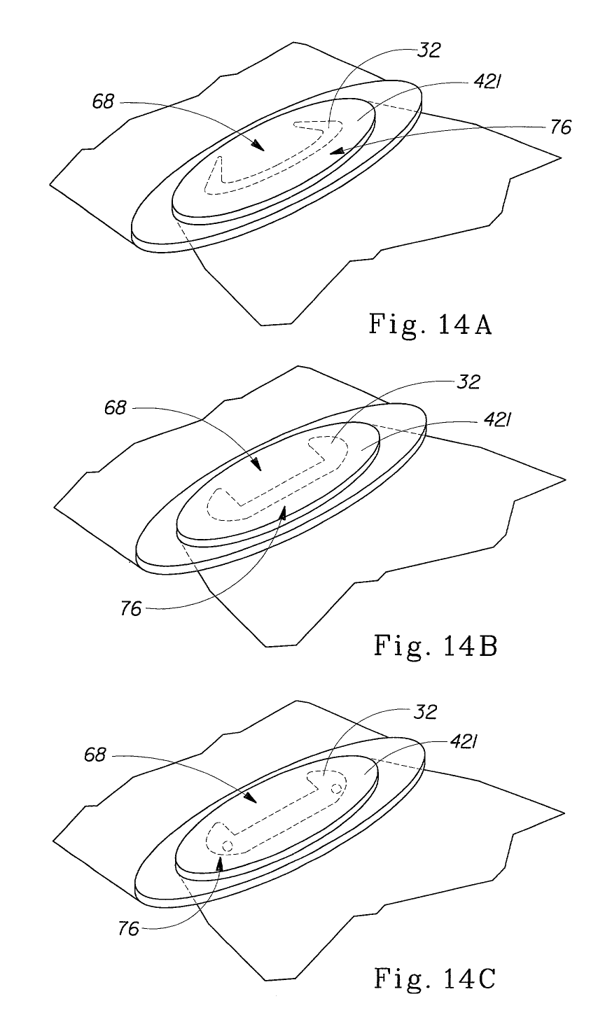

As shown in FIG. 14A through FIG. 14C, a tab stiffening engagement portion 32 of the tab member 421 may have a different width, thickness, modulus, or basis weight than either the grip portion 68 and/or tab load bearing portion 76. Preferably, the tab grip portion 68 includes at least one portion having a lower basis weight than at least one portion of the tab stiffening engagement portion 32. Preferably, the ratio of basis weight in the grip portion 68 to the stiffening engagement portion 32 is less than about 0.8 and more preferably less than about 0.5. Preferably, the stiffening engagement portion 32 is integral with the tab load bearing portion 76 or the slot load bearing portion 67. The slot member may also have a slot stiffening engagement portion (not shown) with properties as disclosed herein for the tab stiffening engagement portion 32.

Slot Members

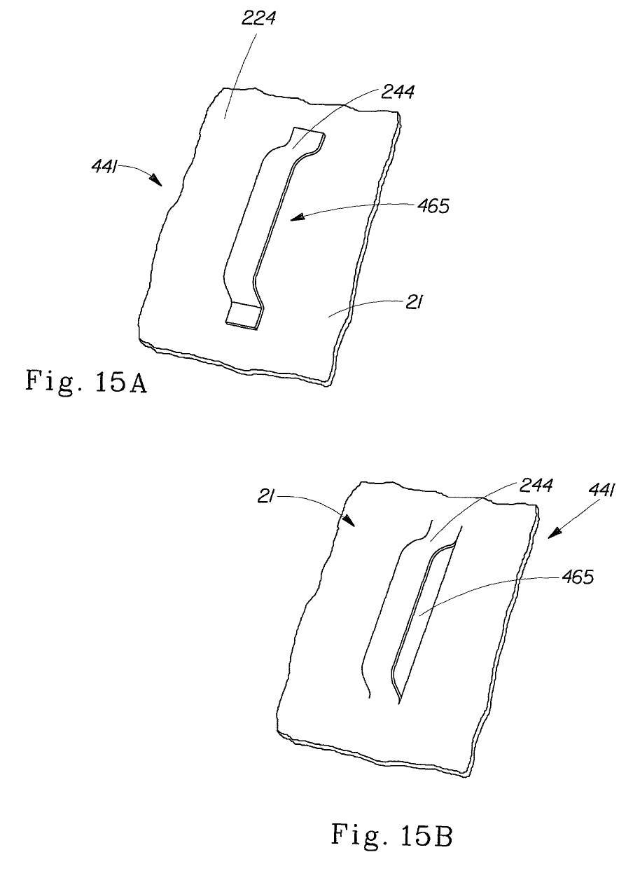

The structure of the slot member opening may vary. The slot 461 may be an opening such as a hole formed by the removal of material. The slot 461 may alternatively include a slit, which is defined as a slot 461 having essentially no gap other than that left by a cutting process. As shown in FIG. 15A and FIG. 15B the slot 461 may also include a loop 465, which is defined as an opening under which a tab member 421 is passed to engage the tab member 421 and slot member 441. The loop 465 may be formed by attaching a strip of material 244 to the article 21 as shown in FIG. 15A. Alternatively, the loop 465 may be formed by at least two slits such as the slot member 441 shown in FIG. 15B formed by cutting a strip of material 244.

As shown in FIG. 16, the slot member 441 may include at least one slot 461 with a slot length S and a slot width SW. The slot 461 is the portion of the slot member 441 into which the tab member 421 (FIG. 9) may be inserted. The slot length S may be less than the unbent tab member length T (FIG. 9). The slot member may include more than one slot to create additional retention capability or adjustment capability. The slot member 441 may also include a slot member width W, at least one longitudinal end 45, a slot central region 61, a slot inboard portion 64, a slot outboard portion 66, a line of attachment 72, a slot stiffening member 77, and a slot grip portion 69.

Slot Member Design

As shown in FIG. 17, a slot member 441 may have at least one load bearing portion 67 (LBP). The load bearing portion 67 is defined as the portion of the slot member 441 that is immediately adjacent the slot 461. Preferably, the portion of the slot member 441 within about 0 to about 15 mm, of either side of the slot 461 may be considered the load bearing portion 67. A slot gripping portion 69 is defined as any remaining slot member 441 outside the load bearing portion 67. Although there may be no difference in material properties or structure between the load bearing portion 67 about the slot 461 and the grip portion 69, both the load bearing portion 67 and the grip portion 69 may have different material properties or structure within their respective portions. Preferably, the load bearing portion 67 and grip portion 69 have different material properties, and/or structural differences. The material and/or structural differences between the load bearing portion 67 about the slot 461 and the grip portion 69 may include a gradual transition in properties. The load bearing portion 67 and the grip portion 69 may both also have different material properties or structure within their respective portions.

For example, as shown in FIG. 17, the slot load bearing portion 67 may include a slot structure with a plastic such as polypropylene of a thickness greater than about 0.25 mm about the slot 461. The plastic may be covered by a relatively light, flexible nonwoven. The nonwoven layer or layers may extend beyond the perimeter of the plastic piece. In such an embodiment, the load bearing portion 67 is defined by the portion of the slot member 441 including at least the plastic and the grip portion 69 is any other portion of the slot member 441. In other embodiments, the slot member 441 may be locally weakened for improved flexibility, preferably in the slot central region 61. Methods of weakening the material include scoring, cutting, thinning, bending, heat treating, chemical treating and the like.

The slot member 441 in FIG. 17 may also include a slot member width W, a slot member length L, at least one longitudinal end 45, a slot central region 61, a slot inboard portion 64, a slot outboard portion 66, a line of attachment 72, a slot stiffening member 77, and a slot grip portion 69, a slot load bearing portion longitudinal end width 671, and a slot load bearing portion central region width 672.

Slot material

The slot member 441 may be of any size and/or shape and may be made from any combination of suitable material. The slot member 441 may be made of materials the same as or different from the tab member 421 including plastics, films, foams, nonwoven webs, woven webs, paper, laminates, metals, fibers, fiber reinforced plastics and the like, or combinations thereof. As with the tab member 421, it may be preferred that the materials making up the slot member 441 be flexible. However, the slot member 441 should be stiff enough in the x-direction and/or y-direction so as not to deform and let the tab member 421 disengage under in use fastening forces. The material from which the slot member 441 is made can be reinforced or weakened at certain locations to help provide the desired levels of flexibility and stiffness to the fastening device 41.

Preferred plastics for the slot member 441 include polyester, polypropylene, polyethylene, polystyrene, nylon, and the like. Preferred metals include steel, aluminum, copper, tin, brass, combinations thereof, and the like. Suitable fibers may include natural and/or synthetic fibers.

In order to optimize the body conformity and relative deformation performance of the fastening device, the slot member 441 grip portion 69 and load bearing portion 67 shown in FIG. 17 may include different materials and properties. The materials and properties may also vary within the slot member 441 grip portion 69 and load bearing portion 67.

Preferably, the grip portion 69 is made of material that is thin with a low modulus of elasticity to provide flexibility. Examples include materials with a thickness less than about 1.0 mm, preferably less than about 0.5 mm, and more preferably less than about 0.25 mm. The material may also have a modulus of less than about 1.5 Gigapascals (Gpa), preferably less than 1.0 Gpa, and more preferably less than 0.5 Gpa. The grip portion 69 may have a basis weight less than about 100 gsm, preferably less than about 70 gsm and more preferably less than about 30 gsm.

Examples of preferably grip portion 69 materials include nonwovens such as carded, spunbond, meltblown, spunbond-meltblown-spunbond, and the like. The grip portion 69 may also be a laminate comprising two or more layers of material. Exemplary laminates include nonwoven-nonwoven, nonwoven-film, and the like. Grip portion 69 materials may be integral with load bearing portion 67. Alternatively, the grip portion 69 material may comprise separate pieces of material attached to the load bearing portion 67. Preferably, the entire exterior surface of the slot member 421 is covered in a soft, fuzzy material such as a nonwoven.

Preferably the load bearing portion 67 is made of material that is thin, with a high modulus of elasticity. Preferred load bearing portion 67 material thickness & modulus requirements may vary with slot length in order to meet the body conformity and/or relative deformation objectives of the fastening device. Shorter slot lengths S (FIG. 16) allow thinner and/or lower modulus materials, while longer slot lengths S require thicker and/or higher modulus materials to deliver generally equivalent relative deformation results. For relatively short slot lengths S of less than about 6 cm relatively thin materials and/or materials having low modulus may be used. The thickness of the slot is measured in the z-direction as shown in FIG. 2B. The load bearing portion 67 preferably has a thickness less than about 0.5 mm and more preferably less than about 0.25 mm. The load bearing portion 67 preferably has a modulus greater than about 200 MPa, and more preferably greater than about 500 MPa.

For slot lengths S greater than about 6 cm, thicker and/or higher modulus materials are preferred. The load bearing portion 67 preferably has a thickness greater than about 0.3 mm and more preferably greater than about 1 mm. The load bearing portion 67 preferably has a modulus greater than about 500 MPa, and more preferably greater than about 1000 MPa. For load bearing portion 67 materials of extremely high modulus, such as steel, the material thickness may be reduced to less than about 0.2 mm.

The grip portion 69 and load bearing portion 67 may have a different basis weight (weight/unit area). Preferably, the grip portion 69 includes at least one portion having a lower basis weight than at least one portion of the load bearing portion 67. Preferably, the ratio of basis weight in the grip portion 69 to that in the load bearing portion 67 (BW₆₉/BW₆₇) is less than about 1, less than about 0.25 and more preferably less than about 0.1.

Preferred Slot Member Load Bearing Portion Parameters

The stiffness within the slot load bearing portion 67 may be generally constant in the x-direction, y-direction, and/or z-direction. In a preferred embodiment, the stiffness within the load bearing portion 67 may vary along the x- and/or y-directions. Stiffness may be varied by varying plan view area, thickness, basis weight, and/or modulus of the slot load bearing portion 67.

In one preferred embodiment shown in FIG. 17, the plan view area of at least a portion of the load bearing portion 67 is varied in the xy-plane. This may be achieved by preferably having a slot load bearing portion longitudinal end width 671 on at least one side of the slot member 441 wider in the x-direction near at least one slot longitudinal end 45 of the slot member 441 than a slot load bearing portion central region width 672 located approximately in the slot central region 61 of the slot member 441. Preferably, the width ratio of the slot load bearing portion longitudinal end width 671 to the slot load bearing portion central region width 672 is greater than about 1.0, greater than about 1.25, and preferably greater than about 2.

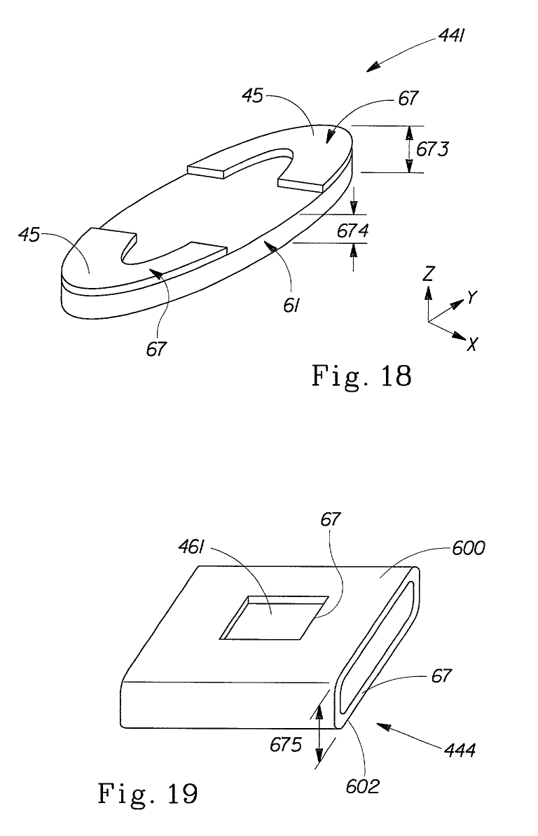

As shown in FIG. 18, at least a portion of the slot load bearing portion 67 may be varied along the y-direction. For example, the z-direction end thickness 673 near at least one slot longitudinal end 45 may be thicker than a central thickness 674 in the slot central region 61. Preferably, the thickness ratio of the end thickness 673 to the central thickness 674 is greater than about 1.0, greater than about 1.1, and preferably greater than about 2.

In another preferred embodiment shown in FIG. 18, the modulus of elasticity of at least a portion of the load bearing portion 67 may be varied along the y-direction and/or the x-direction. Preferably, the at least one slot longitudinal end 45 has a modulus higher than the modulus in the slot central region 61. Preferably, the modulus ratio of the longitudinal end modulus to the slot central region 61 modulus is greater than about 1.0, and preferably greater than about 3, and more preferably greater than about 10.

As shown in FIG. 18, the slot member 441 and/or the load bearing portion 67 are preferably relatively thin in the z-direction, relatively narrow in the x-direction, and/or relatively long in the y-direction. The preferred z-direction thickness 674 may be less than about 5 mm, less than about 3 mm, less than about 1 mm, and/or less than about 0.5 mm. Preferred x-direction slot member width W as shown in FIG. 17 are less than about 40 mm, less than about 30 mm, less than about 20 mm, less than about 15 mm. Preferred y-direction length L (FIG. 17) is more than about 20 mm, more than about 30 mm, more than about 50 mm, more than about 60 mm. Preferably, the slot member 441 and/or the load bearing portion 67 have a width to thickness ratio of more than about 5, more than about 10, more than about 15, more than about 20 or more than about 30. Preferably, the slot member 441 and/or the load bearing portion 67 also have a length to thickness ratio of more than about 10, more than about 40, more than about 70, more than about 100. The dimensions and ratios may apply to either the overall slot member and/or the load bearing portion 67 within the slot member. The slot member 441 stiffness may vary in any direction. The slot member 441 stiffness preferably varies in the y-direction and/or x-direction.

In another preferred embodiment, basis weight (weight/unit area) of at least a portion of the slot load bearing portion 67 is varied along the y-direction and/or the x-direction. Preferably, the basis weight near at least one slot longitudinal end 45 is higher than the basis weight in the slot central region 61. Preferably, the basis weight ratio of the longitudinal end basis weight to the slot central region basis weight is greater than about 1, and preferably greater than about 2, and more preferably greater than about 5.

Preferably, slot member stiffness varies in the x-direction as well as the y-direction. For example, the basis weight, thickness, and/or modulus of the slot load bearing portion 67 may vary in the x-direction, y-direction, and/or z-direction. The preferred basis weight, thickness, and/or modulus ratios described for longitudinal variations also apply for lateral variations.

Special Additional Consideration for Housing Style Slots

One alternate in plane engagement fastening device is a housing style tab and slot formation. A housing style slot members 444 as shown in FIG. 19, may be optimized for body conformity and relative deformation as previously described by minimizing the material forming the housing thickness 675 of the slot member 444 and minimizing modulus of the materials forming the slot member 444.

For example, a first plate 600 and a second plate 602 may have a substantially different thickness and/or modulus. In one embodiment the first plate 600 may be made from plastic film, while the second plate 602 may be made from a flexible nonwoven. The resulting housing slot member 444 has improved body conformity characteristics than a housing slot made from just one material, while maintaining the ease of engagement and load bearing characteristics desired in a housing slot member 444. The load bearing portion 67 may be reinforced around the slot 461 opening for added strength for improved relative deformation. The housing type slot members 444 shown in FIG. 19 may include an additional openings in the x-direction, for tab member 426 insertion and provide additional retention and/or adjustment capacity.

Details on “Projectile & Receptacle” In Plane Engagement Fastening Devices

The male and female members of a projectile and receptacle in plane engagement fastening device can take various forms. Two projectile and receptacle fastening device forms include rod and socket, and ball and socket. The male projectile member (rod or ball) includes a projectile that extends from a surface. The female receptacle member (socket) includes a receptacle that interlocks with the male member.

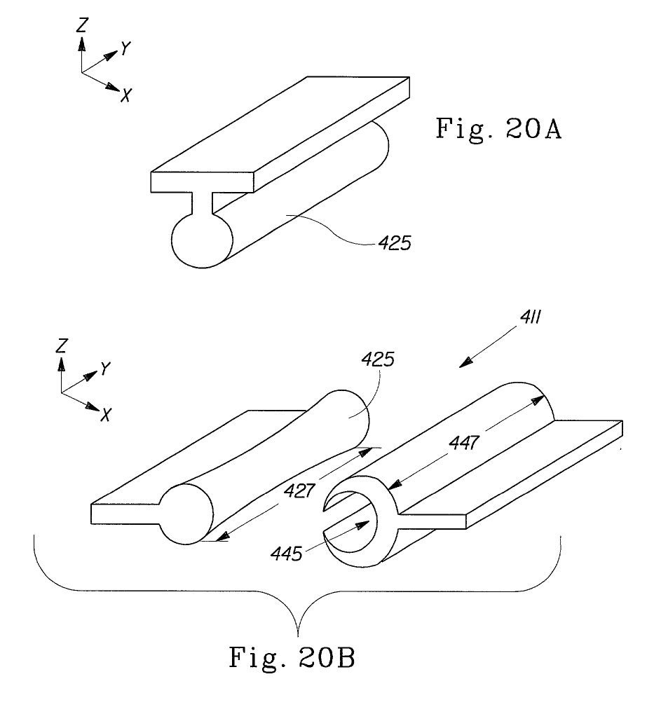

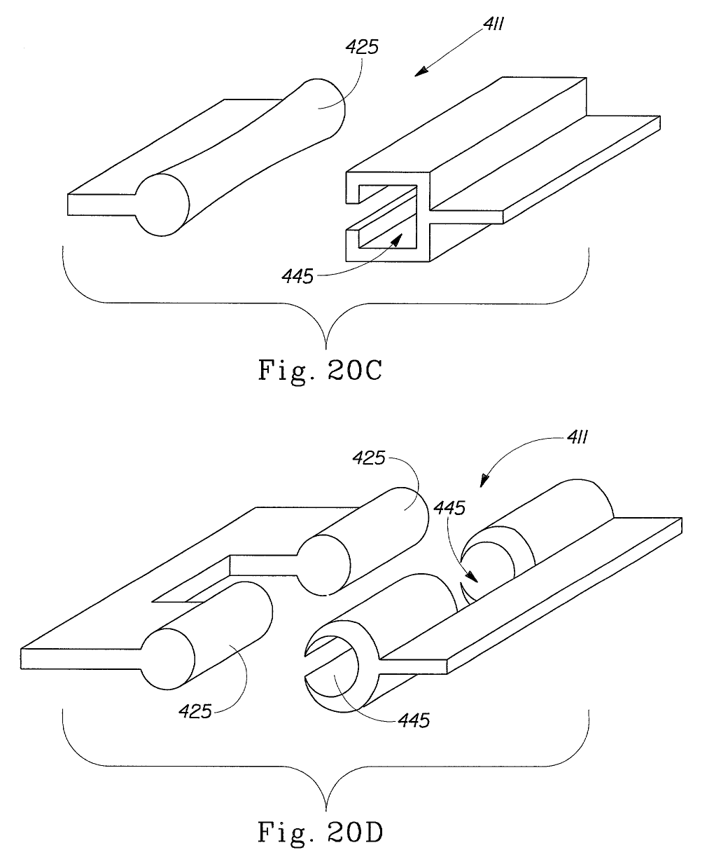

Depending on the design of the specific projectile and receptacle and how it engages, the projectile and receptacle fastening device could be an in plane engagement fastening device or an out of plane engagement fastening device. For example, a projectile and receptacle fastening device 411 as shown in FIG. 20A may be designed to be used as an out of plane engagement fastening device wherein the projectile 425 is pushed into the receptacle 445 in the z-direction causing elastic deformation of the receptacle 445 until it form back around the projectile 425. Alternatively, the projectile and receptacle fastening device 411 may be used as an in plane engagement fastening device by sliding the rod into the end of the socket in the y-direction. Fastening devices designed for either means of fastening are considered in plane engagement fastening devices.

Rod & Socket

A rod and socket projectile and receptacle fastening device is shown in FIG. 20A through FIG. 20D. The projectile 425 and receptacle 445 may have various shapes, sizes and cross sections including sphere, rod, pyramid, cube, cylindrical, circular, triangular, square, oval, and the like. The projectile 425 may extend into the z-direction from any location on the xy-plane of the fastening device surface as shown in FIG. 20A. Alternatively the projectile 425 may extend laterally in the x-direction and/or y-direction as shown in FIG. 20B. The projectile 425 size and shape are designed to interlock with a complimentary receptacle 445. However they need not be a match between the cross-section of the projectile 425 and the receptacle 445 as shown in FIG. 20C provided the fastening device is otherwise designed to remain fastened as needed. As shown in FIG. 20B, a projectile length 427 and a receptacle length 447 may be the same or different lengths for a particular fastening device 41 application.

In order to improve the body conformity performance of the fastening device 411, it may be preferable to vary the projectile 425 and/or the receptacle 445 projectile dimensions along the x-direction and or the y-direction. For example as shown in FIG. 20B a small variation on the projectile 425 has a discontinuity in the y-direction. Alternatively, the projectile 425 and/or the receptacle 445 may be segmented as shown in FIG. 20D. As shown in FIG. 20D the rod 422 and socket 442 may be segmented or combined such that a series of fastening devices are used to fasten the article and/or to create a in plane engagement fastening device 411.

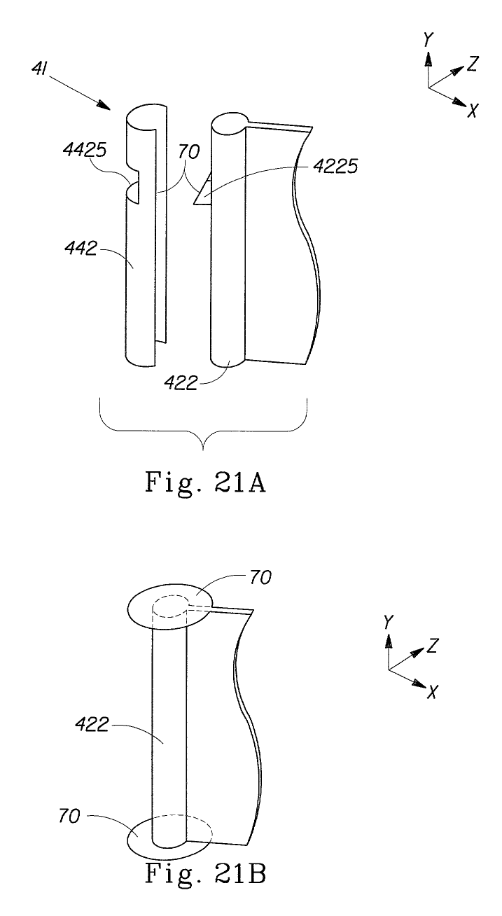

An in plane engagement fastening device 41 may include a retaining element. The retaining element provides added resistance to disengagement of the fastening device 41 to shear loads in the +x-direction, −x-direction, +y-direction, the −y-direction and combinations thereof. One example of a retaining element 70 is shown in FIG. 21A and FIG. 21B.

In order to improve the shear load capacity of any in plane engagement fastening device 411, at least one retaining element 70 may be added as shown in FIG. 21A and FIG. 21B. The retaining element may be added to the projectile 425 and/or the receptacle 445. Alternatively, the retaining element may be any of the previously described tab retaining elements. Preferably, the retaining element 70 is added such that the in plane engagement fastening device resistance to shear mode disengagement is at least more than about 50 g, more than about 100 g, more than about 500 g, more than about 1000 g in at least one x-direction (positive or negative) and at least one y-direction (positive or negative). The retaining element 70 may include a portion that extends from a surface of either projectile 425 and/or the receptacle 445 to provide additional resistance to disengagement.

FIG. 21A shows the projectile as a rod 422 that is inserted into the receptacle portion, which is a socket 442, to fasten the fastening device 411. A latch 4225 and a hole 4425 act as the retaining element 70. The latch 4225 is located on the rod 422 and the hole 4425 is located on the socket 442. The rod 422 slides within the socket until the latch 4225 enters the hole 4425 in the socket 442. Once latched, the fastening device 41 may carry a significant in use load in any direction without disengaging. However, a relatively small manual manipulation of the latch 4225 would allow the fastening device 41 to easily disengage. Alternatively, the latch 4225 could be located on the socket 442 and the hole 4425 could be located on the rod 422. FIG. 21B shows an embodiment of the retaining element 70 as two caps to prevent shear load disengagement. In other selected embodiments, the fastening device may include a plurality of retaining elements such as sockets, latches, holes, and the like.

Preferred Materials and Properties for Projectiles and Receptacles

The projectile 425 and/or the receptacle 445 may be made of any material herein disclosed as suitable for a tab member 42 and slot member 44 respectively. The projectile 425 and/or the receptacle 445 is preferably relatively thin in the z-direction, relatively narrow in the x-direction, and relatively long in the y-direction. Therefore, the projectile 425 and the receptacle 445 may preferably have the same z-direction, x-direction, and y-direction dimensions as disclosed herein as suitable for a tab member 42 and slot member 44 respectively.

Fastening Device Combinations

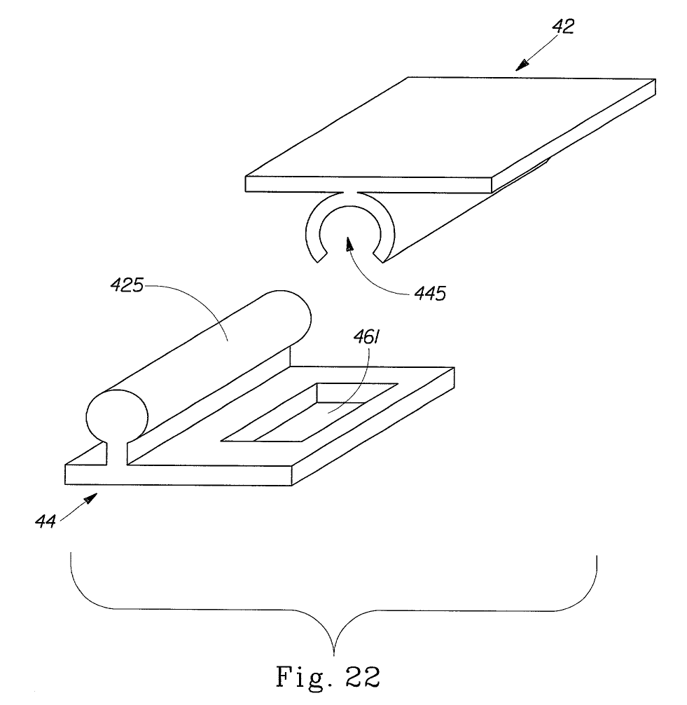

A tab member 42 and/or slot member 44 may be combined with a projectile 425 and/or the receptacle 445 to form one fastening device as shown in FIG. 22 to provide improved resistance to z-direction loads. As shown in FIG. 22, the tab member 42 includes a receptacle 445 which is passed through a slot member 44 slot 461. The slot member 44 includes a projectile 445, such that the projectile 445 engages with the receptacle 445 to form a connection which has improved load carrying capacity in at least the x-direction due to the projectile 425 and receptacle 445 fastening device 41 and in the y-direction and/or z-direction due to the tab member 42 and the slot member 44 fastening device 41. Many such combinations are possible to create the desired balance of shear & peel disengagement resistance, flexibility, and ease of engagement.

The fastening device 41 may be used alone or in conjunction with other fastening means such as hook and loop fasteners, tape fasteners, snaps, buttons and the like to provide different fastening characteristics. For example, the fastening device 41 may include a feature such as the hook material typically used with hook and loop type mechanical fasteners on the tab member or slot member. This hook material may be used to provide the diaper 20 with a disposal means (disposal fastening device) for fastening the diaper 20 in a configuration convenient for disposal. The disposal fastening device may include a tape tab or a hook and loop fastener. Further, a secondary fastening means may be used to adjust the article fit or increase the strength of the fastening device's 41 connection between the first waist region 36 and the second waist region 38.

General Test Guidelines

All testing is to be conducted in standard conditions, specifically in a room held at 50%±2% relative humidity and 73±2° F. All materials to be tested are to be pre-conditioned at these standard conditions for a period of at least 2 hours (and preferably 24 hours) prior to testing.

Thickness is to be measured under a 0.6±0.03 psi load (4.136854±0.2 kilopascal) between two flat, parallel surfaces using ASTM method D5729 and the standard conditions listed above. The circular presser foot size may be reduced to as small as 2 mm diameter and equipment modified to result in a 0.6±0.03 psi load (4.136854±0.2 kilopascal) as needed to accommodate measuring small test samples, or small variations within a test sample. The in plane engagement fastening device should be measured sufficiently to determine z-direction thickness variations in the x-direction and/or y-direction.

Basis weight is to be measured using any suitable method of determine mass per unit area. Suitable methods include EDANA 40.3-90. Smaller test areas may be used if needed to measure basis weight variations within the test sample (fastening device). In any case, a sample of known area is weighed. The result is determined by dividing the mass of the sample by the area of the sample. The in plane engagement fastening device should be measured sufficiently to determine basis weight variations in the x-direction and/or y-direction.

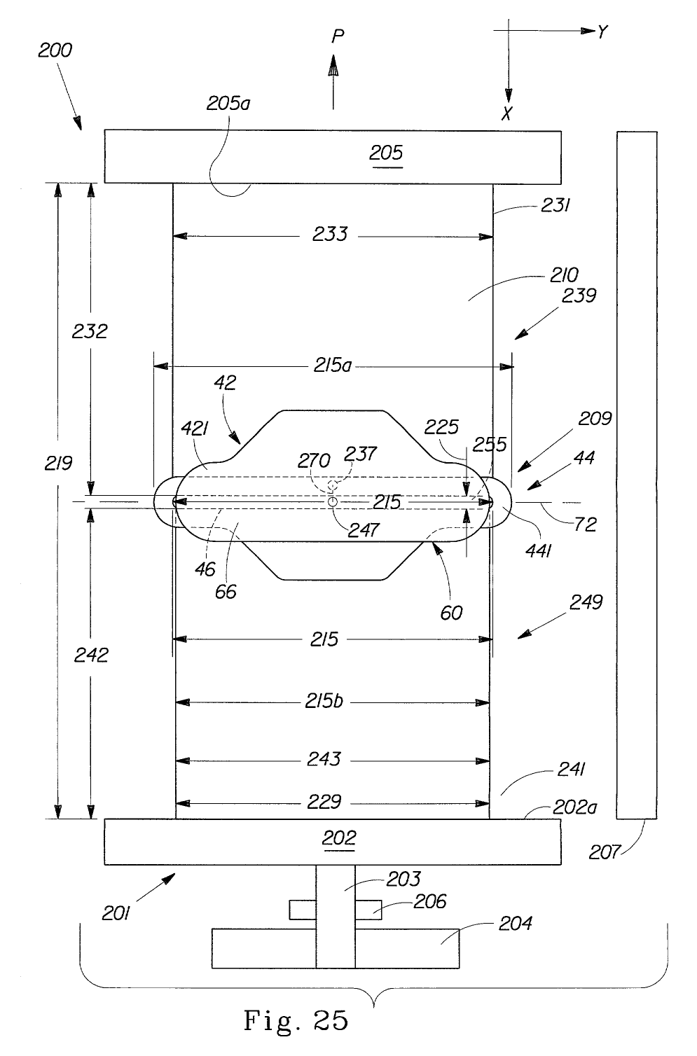

Product extension under load test data results may be obtained for absorbent articles such as the diaper 20 shown in FIG. 2 by using the following test method. Fasten the fastening device 41 on one side of the diaper. Cut the crotch region 37 along the transverse axis 110 as shown in FIG. 2. As shown in FIG. 1, measure the initial waist circumference 352 to the nearest millimeter without applying a tensile load (or any load) on any contracted article components. The measurement is taken beginning at the line of attachment 72 on the unfastened slot member 441 as shown in FIG. 16, around the waist circumference 352 (FIG. 1), to the line of attachment 72 on the tab member 421 (FIG. 9). Using a test apparatus as shown for relative deformation in FIG. 25, clamp the not yet fastened female fastening member 44 in upper clamp 205. Clamp the not yet fastened male fastening member 42 in the lower clamp 202. Upper clamp 205 and lower clamp 202 are to be wide enough such that no portion of either fastening member protrudes from either y-direction end of either clamp. Each fastening member is to be centered in its respective clamp. The line of attachment 72 for each member of the unfastened fastening device in the top clamp 205 and the bottom clamp 202 should generally align with the top clamp edge 205a and the bottom clap edge 202a. Apply the load under which extension is to be measured. Measure the extended length under load, that is distance between upper clamp edge 205a and the bottom clamp edge 202a, and record to the nearest millimeter. The measurement is to be taken from the y-direction center of the female fastening member 44 to a point directly vertically downward to the male fastening member 44. Calculate the percent extension as 100*(extended length under load−initial waist hoop circumference)/(initial waist hoop circumference). Remove the diaper 20 from the clamps. Re-measure a final waist hoop circumference in the same manner as initial waist hoop circumference was measured. Calculate the percent relaxation as 100*(final waist circumference−initial waist hoop circumference)/(initial waist hoop circumference).

The shear load capability of an in plane engagement fastening device is measured on absorbent articles such as the diaper 20 shown in FIG. 2 by using the following test method. A test apparatus similar to that shown for relative deformation shown in FIG. 25 may be used, but with upper clamp 205 affixed to a measuring device (not shown) capable of reading load to at least the nearest gram. Guidance on leader materials, lengths, and attachment techniques cited in the relative deformation test procedure are to be followed for the shear load testing. When testing for shear load in the y-direction the leaders are attached at the longitudinal or y-ends of the test sample such that the leaders extend in opposite directions from the fastening device. These attachments to the fastening device should be stronger than the shear load of the fastening device in the y-direction and not interfere with the test results. The load is applied in the ±y-direction of the fastening device 41 such that the male member 42 and the female member 44 are pulled in opposite directions. The test method includes affixing a female leader at least as wide as the female member to the female member in the direction to be tested. If the female member is to be tested in the x-direction, the leader is attached to the x-direction ends of the female member. If the female member is to be tested in the y-direction, the leader is attached to the y-direction ends of the female member (e.g., the x- or y-direction width). A male leader at least as wide as the male member is also affixed to the male member in the direction to be tested (e.g., the x- or y-direction width). Fasten the fastening device. Each leader is to be centered in its respective clamp. Secure the female leader in upper clamp 205. Secure the male leader in lower clamp 202.

The test may be run in the x-direction as shown in FIG. 25 or in the y-direction (not shown). The direction to be tested (ie, the x- or y-direction) should be within about 1 degree of vertical for that direction. A slow and steady load is applied to the lower clamp 202 until the male member 44 disengages with the female member 42. A slow and steady load is about 100 mm per minute. Record the peak load which occurred during testing.

Modulus is measured using ASTM D638-98 and or ASTM D882. The specimens are cut using ASTM D412 Die C, with samples tested in both machine and cross directions. Report the modulus of elasticity using the tangent slope a low stress using ASTM D638-98.

Body Conformity Test Procedure

The body conformity test method measures the generally compressive load required to deflect a fastening device sample through a range of bending about the x-axis and or the y-axis. The body conformity test method provides a means for measuring the bending capabilities of the combined first and second fastening members of an in plane engagement fastening device. A high body conformity test result indicates good flexibility and is therefore desirable. The flexibility allows the fastening device to conform to the contour of a wearer and provide comfort throughout a range of motions and activities by the wearer.

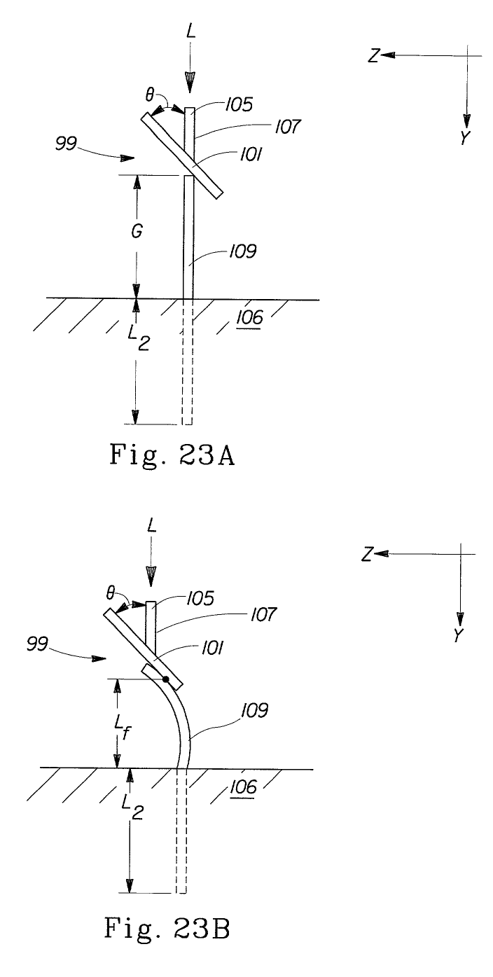

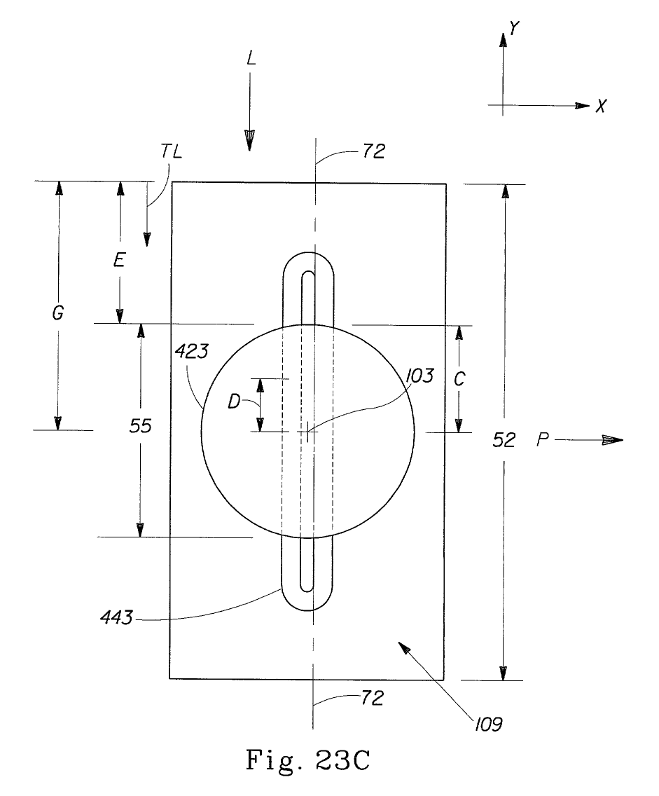

A test fixture 99 for measuring body conformity is shown in FIG. 23A and FIG. 23B. The test fixture includes a foot 101, a measuring device 107, and a test sample holder 106. A body conformity test method measures the generally compressive load required to deflect a fastening device sample 109 through a range of bending deflection using the foot 101 that comes down on the sample 109 with a load L applied through foot 101 at an angle θ of about 45 degrees to the load L.

The body conformity test method may be used to measure the bending capabilities of the combined first and second fastening members of the fastening device sample 109. One way to do this is to have the foot 101 come down vertically on the fastener with the foot at a 45 degree angle θ to the direction of travel. The range of deflection loads applied to the sample 109 by the foot 101 may be between about 0 grams and about 1.5 kilograms (kgf). The method measures the compression of the fastening device sample 109 as a function of the load in grams-force applied. The resulting data is used to calculate a body conformity (percent deflection per kilogram of load). The higher the deflection for a given load, the higher the body conformity. The test is run until the fastening device sample 109 reaches a maximum load of 1500 grams-force or 50% of the combined fastening device sample length C (FIG. 23C), whichever comes first. The combined fastening device sample length C is the portion of the length of the fastening device test sample 109 in which both the male fastening member 423 and female fastening member 443 overlap in an attached configuration.

FIG. 23C shows a fastening device test sample 109 as it relates to the test procedure. The fastening device sample 109 preparation begins with removing the fastening device 41 from the article. If the fastening device 41 is integrated into the article, the fastening device 41 may be cut out of the article along with any portions of the article that are related to the performance of the fastening device 41. Some amount of material around the fastening device sample 109 may be maintained so as not to compromise the fastening device sample 109. One example is leaving material around a buttonhole. This extra material should be of equal length on both ends of the fastening device sample 109 and should be included in the measurement for gauge length G. The gauge length G is ½ the sample length 52 of the fastening device sample 109 including any added material needed so as not to compromise fastening device 109 when being cut from the article 20. The male and female members of fastening device 41 are connected in a fastened configuration.

The fastening device sample length 52 shown in FIG. 23C is defined as the measurement of the fastened fastening device sample 109 that is perpendicular to a primary direction of load P. Generally, length 52 is also parallel to the load L applied to the test sample 109. This provides consistency over the broad range of fasteners applicable to this method. Length 52 is measured to the nearest 1 millimeter. Fifty percent of this measured length 52 is defined as the gage length G. The center 103 of the fastening device is identified and marked within 1 millimeter. Center 103 is defined as the location along the y-direction coinciding with 50% of sample length 52 and in the x-direction coinciding with the line of attachment 72. An extension E should also be measured to the nearest 1 millimeter. The extension E is defined as that portion of the fastening device sample 109, which is longer than the combined sample length C. The measurement is taken from the outer most edge of the sample 109 along the length 52, to beginning of the combined sample. That measurement is the fastening device sample extension E. The extension E includes any material needed to maintain the fastener integrity along the sample length 52. By definition, combined sample length C equals (G−E).

A down point length D shown in FIG. 23C is then calculated. Down point length D is defined as 50% of combined sample length C. The down point coincides with the y-direction location at which test fixture should stop compressing (unless test fixture stops compressing prior to reaching down point length D due to reaching the prescribed load limit of 1.5 kg).

As shown in FIG. 23A and FIG. 23B, the measuring device 107 may preferably include a computer programmed tensile tester, such as an MTS Alliance RT/1, to accurately and precisely report the load required to move the foot 101 a specified distance at a specified rate. In one embodiment, the foot 101 is a bar that measures about 12 millimeter (mm) wide by about 70 mm long and is about 12 mm thick. The foot is made of steel, with the surface which contacts the fastening device sample 109 polished to a mirror finish. The foot 101 needs to be protected from any scrapes so that the mirror finish is maintained and the fastening device sample 109 may slide on foot 101 during the testing. The foot is connected to a rod 105 that is attached about 15 mm from the back edge of the foot and at a 45° angle. The rod 105 is about 45 mm long at the longest point from the locking collar to the attached foot 101. The rod 105 is designed to fit the tensile tester's top fixture with a locking collar and a cotter pin to minimize any wobble. Test sample holder 106 is designed to fit in the tensile tester's bottom fixture with a locking collar to minimize any wobble. When foot 101 and test sample holder 106 are placed properly in the tensile tester, the center of the rod 105 will be aligned approximately evenly with the center of fastening device sample 109 at contact when viewed from the side as shown in FIG. 23A. The test is designed to begin with the foot 101 just in contact with fastening device sample 109 at a distance G from the sample center point 103 and to terminate either at the load L test limit of 1500 grams force or when the final length Lf equals the down point length D, whichever is reached first. If a fastening device sample 109 cannot be compressed to down point length D before a load of 1500 grams force is reached, final length Lf will not be the same as down point length D.

Testing begins by zeroing the load on the measuring device 107 with the fixtures in place, but prior to placing the fastening device sample 109 in the test fixture 99. The fastening device sample 109 should be tested under a compressive Load L, which is perpendicular to the primary direction of load P and/or parallel to the sample length 52. If the angle of the primary direction of Load P relative to the fastener is less than or equal to 45° to the x-axis, the primary direction of load is defined as being in the x direction to simplify testing.

As shown in FIG. 23A, the fastening device sample 109 should be placed in the test sample holder 106 at the center 103 such that ½ of fastening device sample 109 is above the test sample holder 106 when viewed from the side as in FIG. 23A and such that the foot 101 is centered on the line of attachment 72 when viewed from the surface of the xy-plane as in FIG. 23C. The gripping location of the test sample holder 106 should be precise within about 1 mm of true center 103 of the fastening device sample 109. The fastening device sample 109 should also be centered under the foot 101 and in the test fixture 99. The foot 101 should be lowered so that it is visually touching the fastening device sample 109 and is producing only a very small load, e.g. less than 0.9 grams as shown in FIG. 23A. The crosshead position of the tensile tester is then zeroed and the test is run by moving foot 101 down at a rate of 100 millimeters/minute to apply load L. The test continues as fastening device sample 109 deflects under load until the test terminates when either the foot 101 travels to a location corresponding to 50% of length of the combined sample or a load of 1500 grams force is reached.

As shown in FIG. 23C, as load is applied, the foot 101 (FIG. 23A) will travel down a travel length TL, beginning at zero and increasing until the test terminates. When foot 101 just begins to touch the combined sample, travel length TL will equal extension E. Travel length TL will be a maximum length equal to (0.5*C+E) if the foot 101 reaches down point length D before 1500 grams force load is reached. However, if 1500 grams load is reached before the foot reaches down point D, the travel length TL may be less than (0.5*C+E). In either case, travel length TL will equal (G−Lf) at the end of the test and will always equal 0 at the beginning of the test.

The output data is recorded as load L vs. travel length TL with load L recorded for at least about every 0.5 millimeters of travel length TL, and preferably at least every 0.1 millimeters of travel. From the output data, body conformity is calculated and reported in the units of percent compression per kilogram (kgf). If travel distance TL is greater than or equal to extension length E prior to reaching 1.5 kgf load, body conformity is calculated as follows: Body Conformity=(PC2−PC1)/(LPC2−LPC1), where:

PC1=The percent compression at the beginning of the combined sample;

PC2=The percent compression at termination the test;

The percent compression=100*(TL−E)/C;

LPC2=the load, in kgf, recorded at travel distance TL used to calculate PC2;

LPC1=the load, in kgf, recorded at travel distance TL used to calculate PC1.

PC1 should always equal about zero because travel length TL will equal E at the beginning of the combined sample. PC1 may not be exactly zero due to errors in placing sample 109 in the fixture or because data collection frequency does not record a reading at exactly zero. PC1 should be calculated at the first available data point recorded after TL is greater than E. PC2 is by definition between 0% and 50%;

If foot 101 does not travel a distance equal to extension E prior to reaching the 1.5 kg test limit, body conformity is reported as 0%/Kg. The test is to be run on the fastening device 41 in a fastened configuration and on all individual fastening elements (providing individual elements have a combined sample length C greater than about 0.125″ in length) in a fastened configuration. Individual fastening elements having a combined sample length C less than about 0.125″ in length are not measured but the overall fastening device 41 is measured. The lowest body conformity result, whether it is from the fastening device 41 or one of the fastening elements, is reported as the body conformity. Further, if fastening device 41 includes an mechanical assist means, the combined sample is defined as the portion of fastening device 41 in which the male fastening member, female fastening member, and mechanical assist means overlap in a fastened configuration.

The above description applies to measuring a fastening device for bending about the x-axis. The procedure may also be performed for bending about the y-axis. If this is done, sample orientation is turned 90° such that the load from the foot 101 is applied parallel to the primary direction of loads. Body conformity about the x-axis and about the y-axis are reported separately.

Relative Deformation Test Procedure

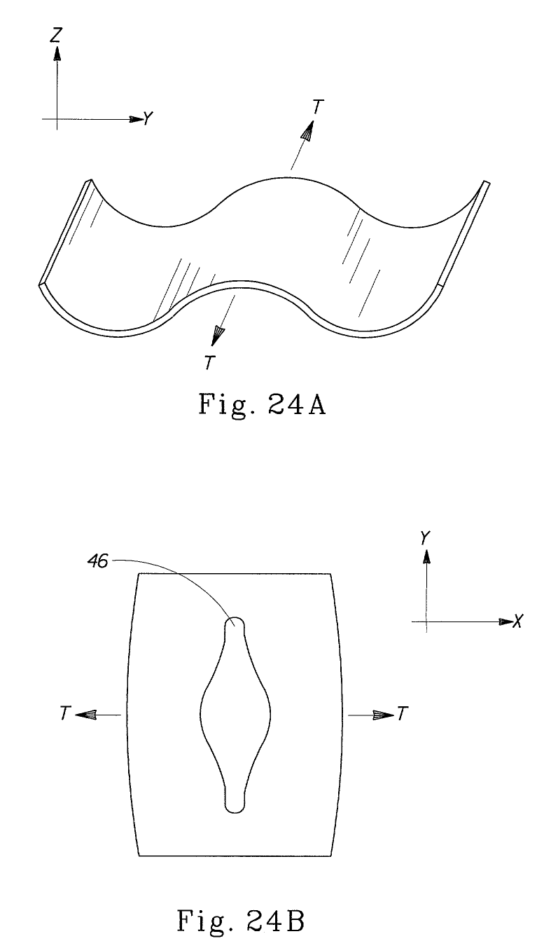

The relative deformation test method was developed to compare fastener performance under load and the fastener's resistance to undesirable deformation. Relative deformation measures the fastening device deformation in the xy-plane under a load in the x-direction. As shown in FIG. 24A, under tensile loading, for the first fastening member 42 and/or second fastening member 44 may ripple, wrinkle, or buckle out of a smooth xy-plane as the fastening device 41 distributes the tensile load. FIG. 24B shows an isomeric view of a slot deforming under tension. In order to quantify the fastener deformation under a tensile load, the relative deformation test was developed to compare fasteners. The relative deformation test measures the deformation of at least one fastening element relative to the initial length of the fastening element at a selected load. The relative deformation is a comparative measurement of the fastening device 41 deformation under a tensile load. Relative deformation may be described as a way to quantify the deflection of the fastening device out of the x-y plane when the fastening device is in tension. Under tension the fastening device may “buckle” as shown in FIG. 24A and open the slot 46 as shown in FIG. 24B. Buckling reduces the smooth aesthetic look of the article and in some cases can cause the fastening device to disengage.

Deformation of the fastening device, as shown in FIG. 24A, may reduce the aesthetic appeal of the article, lead to skin marking, or result in diaper 20 leakage. Therefore, a lower relative deformation in the xy-plane is desired to maximize the load bearing capacity of the fastening device 41. The relative deformation is the percent relative deformation (RD) per kilogram of load applied (% RD/kg). A low number indicates the sample does not deform as much under a tensile load T, as another fastening device with a higher relative deformation.

The relative deformation test method may be used to measure relative deformation of IPE fasteners. To determine relative deformation, the fastening device 41 is tested from about 0 grams up through a maximum tensile load in the xy-plane of approximately 2.4 kilograms (kgf), or 25% relative deformation, whichever comes first. Deformation of fastening device 41, fastening device length, and load applied are used to calculate relative deformation.

A deformation test apparatus 200 is shown in FIG. 25. The deformation test apparatus 200 is a device that will allow a deformation test sample 209 to be clamped securely at one end and freely suspended at the other end so as not interfere with the test results. The deformation test apparatus 200 includes a bottom clamp 202, a top clamp 205, a weight rod 203, a bottom plate 204, weights 206, and a deformation measuring device 207.

The top test sample component 239 and/or the bottom test sample component 249 may be any in plane engagement fastening device component such as the first fastening member or the second fastening member. FIG. 25 shows the top test sample component 239 as a slot member 441 and the bottom test sample component 249 as a tab member 421. The deformation test sample 209 in FIG. 25 includes a top test sample component 239 and a bottom test sample component 249 and a deformation sample length 219.

Preferably, during testing the top test sample component 239 is the female fastening member 44 and suspended from the top clamp 205. The top test sample component 239 may include a top leader 231. The top leader 231 has a top leader length 232 and a top leader width 233. The top test sample also may include a sample slot 255 with a sample slot length 215 and a sample slot width 225.

The bottom test sample component 249 is preferably the male fastening member 42 is attached to the bottom clamp assembly 201. The bottom test sample component 249 may include a bottom leader 241. The bottom leader 241 includes a bottom leader length 242 and a bottom leader width 243.

As shown in FIG. 25, the deformation sample length 219 is defined as the external measurement of the combined test sample 209, including the top leader 231, bottom leader 241, and fastening device 41 in a fastened configuration. The length is measured in primary direction of load P. The primary direction of load P is the direction of the load as it passes through the fastener as intended during use. If a load passes through fastener with more than one directional component, the primary direction of load P is defined as the direction of the larger force component. If the angle of load relative to the fastener x-direction is less than or equal to about 45°, the primary direction of load P is defined as the x-direction.

As shown in FIG. 25, the top clamp 205 is preferably wider than the top leader width 233 and strong enough to grip the leader while holding 2600 grams without slippage. The bottom clamp 202 is preferably wider than the bottom leader width 243 and strong enough to grip the leader while holding 2600 grams without slippage.

Top clamp 205 is secured to anything capable of holding it securely under load of at least 2600 grams while allowing sample 209 to hang freely from top clamp edge 205a and unobstructed directly vertically downward from top clamp 205. Top clamp is secured such that edge 205a is within about 1 degree of horizontal.

A bottom clamp assembly 201 is assembled by securing bottom clamp 202 to weight rod 203 and bottom plate 204 in a manner which will allow loads of up to at least 2600 grams to be supported by bottom plate 204. Bottom clamp assembly 201 is to be designed and assembled such that weight rod 203 will hang generally vertically downward from bottom clamp 202 when test sample 209 is clamped into top clamp 205 and bottom clamp assembly 201 is clamped onto test sample 209. Bottom clamp assembly 201 should be weighed and recorded. The bottom clamp assembly 201 is used as the first increment of load upon the deformation test sample 209. Bottom clamp assembly 201 is to be constructed to have a mass of about 204 grams.

The weights 206 are preferably of a type that fit on the weight rod 203 and rest on the bottom plate 205. The weights 206 are preferably calibrated. Preferably, the weights 206 will include five 100 gm weights, six 200 gm weights, and one 500 gm weight.

The deformation measuring device 207 may be a digital micrometer that is calibrated and reads in millimeters to two decimal places. An exemplary measuring device 207 is a Mitutoyo Model CD-6″ C.

A test sample 209 is prepared for testing in deformation test apparatus 200. If fastening device 41 is attached to an article 20, fastening device 41 is preferably removed from the article 20 in such a manner that existing article material is used as top leader 231 and/or bottom leader 241. In order for this to be done, there must be sufficient article material present to create leaders of the needed sizes as described below. If there is insufficient material to use as leaders or fastening device 41 is a separate component (ie, is not provided with an article 20), then top leader 231 and/or bottom leader 241 may be created from a nonwoven web. Thus, a nonwoven web may be attached to fastening device 41 to create leaders 231 and 241, or a nonwoven web may be used to extend material from the article which is already attached to fastening device 41 yet form suitably sized leaders 231 and 241. A particularly preferred nonwoven for use as the nonwoven web is a spunbond nonwoven made of polypropylene fiber, style number 088 MLPO 09U, as available from BBA of Simpsonville, S.C.

The top leader 231 and bottom leader 241 are designed to apply the load to the test sample 209 in the primary direction of load and in line with the anticipated use of the fastening device 41. Thus, the leaders 231 and 241 should allow fastening device 41 to deform under load in a manner that mimics how the fastening device 41 would behave if attached to the article 20. Any added nonwoven should be attached directly to fastening device 41 in a manner that does not substantially interfere with the engagement or strength of the test sample 209. The attachment of the nonwoven should be strong enough to assure that as the test sample 209 deforms under load the leaders will remain affixed. One particularly suitable approach for joining added nonwoven to fastening device 41 and/or other article material is to secure added nonwoven with a flexible adhesive double-sided tape such as 3M Transfer Adhesive, type #1524.

If the top test sample component 239 includes a sample slot 255, it has been found that looping the nonwoven through the slot 255 and adhering the nonwoven to a portion of the top test sample component 239 and/or onto the nonwoven itself with double-sided tape can reliably secure the nonwoven to the appropriate portion of the top test sample component 239. Other suitable approaches to securing added nonwoven include sewing, hot melt glue, etc., as long as the approach allows the test sample 209 to engage and function. Any addition nonwoven added and the approach to join it to test sample 209 preferably do not interfere with the function of the test sample 209 by significantly strengthening or weakening the sample 209.

As shown in FIG. 25, top leader 231 is attached to the slot member 441. Bottom leader 241 is attached to the tab member 421. If the top test sample component 239 includes a slot 255, the top leader width 233 is preferably from about 2 mm to about 5 mm less than the slot length 215. If the top test sample component 239 does not include a slot 255, the top leader width 233 is preferably about equal to the female member length 215a. The top leader length 232 is preferably twice the female member length 215a plus at least about 25 mm. Bottom leader width 243 is preferably about the same as male member length 215b. The bottom leader length 242 is preferably twice the male member length 215b plus at least about 25 mm. The additional 25 mm of added leader length to each test sample component 239/249 is designed to be the amount of leader placed into the top clamp 205 and bottom clamp 202 respectively. To aid in reliably placing the leader in the clamp, a line may be drawn along the top leader width 233 to show where the top leader 231 will be placed in the top clamp 205. A line may be drawn along the bottom leader width 243 to show where the bottom leader 241 will be placed in the bottom clamp 202. The leader beyond the line (away from the fastening device) would be intended to be placed in the clamp during testing.

A top reference point 237 is marked on the female member 44. A bottom reference point 247 is marked on the male member 42. The reference point locations are chosen such that, as the load is applied, the distance between the two marked reference points can increase. For example, FIG. 25 shows a tab and slot fastening device 41 marked for testing. Top reference point 237 is placed on the slot member 441 above the slot 255. Bottom reference point 247 is marked on the tab member 421. The bottom reference point 247 is preferably at or near the line of attachment 72 as shown in FIG. 25. Using these marking locations, top reference point 237 can move away from bottom reference point 247 if sample 209 deforms as bottom clamp assembly 201 and/or weights 206 hang from sample 209. Further, the reference point locations should be chosen such that it is most convenient to measure the x-direction distance between them. Therefore, reference point locations 237 and 247 are most preferably on the same side of fastening device 41 (e.g. both of the surface facing the viewer in FIG. 25 or both on the surface facing away from the viewer in FIG. 25). Reference point locations are chosen to be within about 1 mm of the y-direction center of fastening device 41.

If a fastening device has more than one fastening element such as the embodiment shown in FIG. 3, (e.g. Two slots spaced along the y-axis designed to engage with two tabs spaced along the y-axis), the test is to be run two ways. First, the test is run for the overall fastening device, marking the reference point locations in the y-direction center of fastening device 41. In an embodiment as shown in FIG. 3, this would be the center between the two slots 461. Second, the test is run on the overall fastening device, but by measuring the x-direction deformations of each individually fastened fastening member. The x-direction deformations are measured under load for reference points located along the fastening member y-direction center for each fastening member combination. As shown in FIG. 3, this would be each tab member 421 and slot member 441 combination. The highest relative deformation result is reported as the relative deformation for the fastening device.

Testing begins by engaging the interlocking fasteners. The top test sample component 239 is then centered into the top clamp 205 such that top leader length 232 is at the test length within about 2 millimeters of horizontal at any point in the y-direction. A light pre-load (ie, less than about 10 grams) is applied by pulling bottom leader 241 downward to be sure that the fasteners are fully engaged. The preload is removed such that the load is equal to about 0 grams. The initial sample deformation length 270 is then measured and recorded as the sample deformation length 270 at zero load. The deformation test sample deformation length 270 is the direct vertical measurement from a top reference point 237 to a bottom reference point 247 which is in line with the primary direction of load P.

A relative deformation normalizing length is calculated using the female member 44. If the female member 44 has a slot 255 as shown in FIG. 25, the normalizing length is equal to the slot length 215. If the female member does not have a slot 255, the normalizing length is equal to the female member length 215a. The normalizing length is measured in a fastened configuration in a direction perpendicular to the primary direction of the load P.

The bottom clamp assembly 201 is clamped onto bottom leader 241 such that bottom leader length 242 is at the test length within about 2 millimeters of horizontal at any point in the y-direction. A new deformation length 270 is measured and recorded with each additional load application. The sample deformation length change is calculated by subtracting the initial deformation length 270 (as measured with a 10 gram pre-load) from the new deformation length 270 (as measured with the 204 gram bottom clamp assembly 201 attached).

A 100 gram weight 206 is added to the bottom plate 204 and weight rod 203. A new deformation length 270 is measured and recorded, along with the total load on sample 209 (now equal to 304 grams given the 204 grams bottom clamp assembly and the 100 gram weight 206). A new deformation length change is calculated by subtracting the initial deformation length 270 at load 10 grams from the deformation length 270 at load 304 grams. This sequence is repeated four more times, each time calculating deformation length change 270 by subtracting initial deformation length 270 at load 10 grams from the deformation length 270 at the new load.

A 200 gram weight is added to the bottom plate 204 and weight rod 203. Deformation length 270 is measured and recorded, along with total load on the sample 209. A new deformation length change is calculated, again by subtracting initial deformation length 270 at load 10 grams from the deformation length 270 at the new load. This sequence is repeated five more times.

A 500 gram weight is added to the bottom plate 204 and weight rod 203. Deformation length 270 is measured and recorded, along with total load on the sample 209. A new deformation length change is calculated, again by subtracting initial deformation length 270 at load 10 grams from the deformation length 270 at the new load. At this point, a total of 2204 grams plus the weight of the bottom clamp, bottom plate 204 and weight rod 203 has been applied to the sample. Thus, the total weight tested is 2404 grams.

The test is preferably done at regular intervals. The weight addition process preferably should not exceed 30 seconds between weight changes, during which time deformation length 270 is to be measured and recorded. If any slippage of leaders 231 or 241 from the grips or if any delamination/separation of materials occurs where any added nonwoven is attached is visually noted, the sample and associated data is to be discarded.

Calculation of Results

Sample deformation is calculated for each load applied after the initial 10 gram pre-load. Sample deformation equals 100*(deformation length change at the prescribed load level))/normalization length. Individual values of sample deformation are plotted on the y-axis of a graph versus the load applied on the x-axis of the graph.

The relative deformation is defined as the highest average slope occurring between a 0% and 25% sample deformation. The average slope is a “Rise/Run” calculation from the 0 load/0 sample deformation point. Thus, for each weight added, the slope equals sample deformation divided by the load applied corresponding with that sample deformation. Since 13 weights have been added, there are 13 average slopes. One of these slopes represents the highest average slope. If the sample deformation does not reach 25%, the relative deformation is defined as the highest average slope occurring between 0% sample deformation and the percent sample deformation reached at the maximum load of 2404 grams. If sample deformation reaches 25% on the first load (eg, 204 grams), the relative deformation is the average slope as calculated using sample deformation corresponding to a load of 204 grams. If sample deformation reaches 25% after the first load (that is, at a load greater than 204 grams) but before the last load, the relative deformation is the average slope as calculated using sample deformation corresponding to the load applied immediately before the load which caused sample deformation to exceed 25%.

Test Results

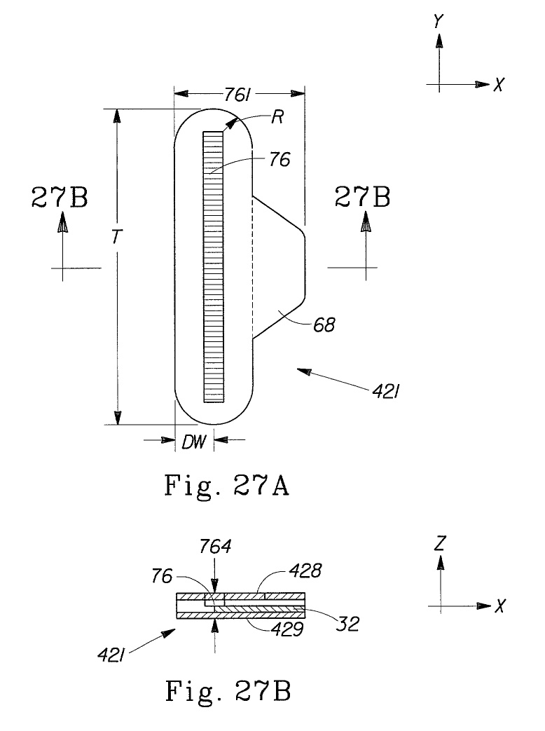

In order to obtain the fastening device 41 capabilities desired, several fastener configurations were tested. The testing was performed to approximate the body conformity of a fastening device 41 by using compressive loading and calculating body conformity. Body conformity was measured as bending about the x-axis as shown in FIGS. 27A-C. Further testing was conducted to assess the “relative deformation” of the fastening device 41 in the xy-plane under tensile loading. Relative deformation of the fastening device 41 was calculated using a tensile load as shown in FIG. 25. FIGS. 23A-C and FIG. 25 are described in detail later with the test procedures.

The following test data generally indicates that some in plane engagement fastening device 41 fastener configurations herein disclosed may be designed to meet a desired fastener engagability, flexibility, alignment, stiffness, and/or combinations thereof. In one example, an optimized in plane engagement fastening device tab and slot configuration may provide an improved combination of conformity through flexibility, and low fastener deformation in the xy-plane.

The in plane engagement fastening device's of the present invention demonstrated the preferred combination of body conformity and/or relative deformation. Body conformity is preferably greater than about 200 percent per kilogram force of load (%/kgf), more preferably greater than about 500%/kgf and most preferably greater than about 1000 %/kgf. The relative deformation is preferably less than about 100 percent per kilogram force of load (%/kgf), more preferably less than about 50%/kgf and most preferably less than about 25%/kgf.

Test Results

The following Table 1 represents a sample of approximate body conformity test results and relative deflection test results for various combined (fastened) in plane engagement fastening devices.

TABLE 1

| | | Approximate | Approximate |
|---|---|---|---|
| | | Body | Relative |
| | | Conformity | Deformation |
| | | (% Deflection/Kg) | (% RD/Kg) |
| Example | | (Higher is | (Lower is |
| No. | Type of Fastener | Desirable) | Desirable) |
| 1 | Shirt Button | 6 | 55 |
| 2 | Depends Button | 20 | 213 |
| 3 | Plastic Buckle | 0 | 0 |
| 4 | Interlocking Rings | 0 | 0 |
| 5 | Snaplock | 0 | 0 |
| 6 | Preferred tab and | 938 | 17 |
| | slot version #1 | | |
| 7 | Preferred tab and | 1382 | 18 |
| | slot version #2 | | |
| 8 | Steel tab and slot | 0 | n/a |

In Table 1 above, examples 1, 2, 3, 4, 5, and 8 are examples of known in plane engagement fastening device fasteners. These in plane engagement fastening device's have excellent load bearing capability but are stiff as shown by the low body conformity values. These in plane engagement fastening device's have a desirable low relative deformation, but this characteristic alone does not provide the preferred capabilities of the fastening device herein disclosed.

Example 1 is a button and buttonhole from a typical men's dress shirt. The button and buttonhole used were taken from the front of a men's shirt manufactured by Van Heusen. The shirt was a pinpoint Oxford, style #11879/a, made in USA and purchased Nov. 4, 1999.

Example 2 is a button and buttonhole from a typical adult diaper available on the market. The button and buttonhole were removed from a Depend Undergarment selected at random from a 36 count package labeled as lot #N98104U3a-1401 and manufactured by Kimberly Clark Inc, Wisconsin.

Example 3 is a plastic buckle including a housing style slot and movable retaining element. Example 3 includes a “Center Release, Fits 1″ Strapping, Style #1105, manufactured by Strapworks of Lansing, Iowa.

Example 4 is a pair of interlocking metal rings including a 1″ long interlocking nickel buckle type #303, ” manufactured by EZ International of Saddle Brook, N.J.

Example 5 is a snap lock plastic buckle including a slot and which interlocks with a non-movable retaining element from Jontay of Aiken, S.C., style #4561 Navy.

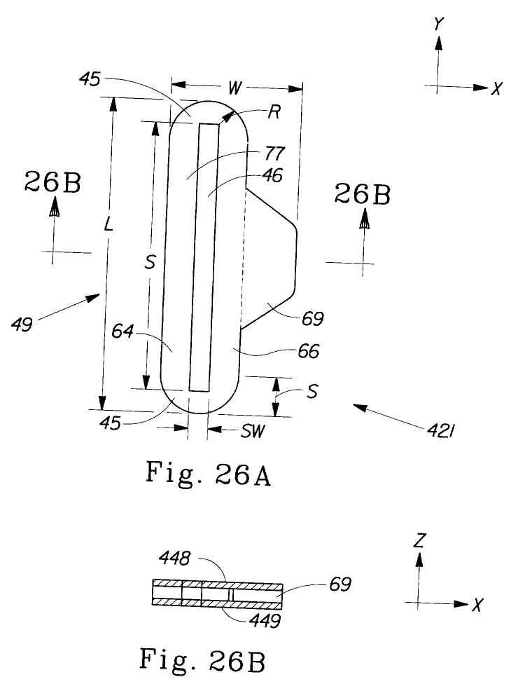

Example 6 is a preferred tab and slot fastening device. The slot member 441 as shown in FIG. 26A and FIG. 26B includes a slot stiffening member 77 made of one layer of about 0.762 mm thick (z-direction) high impact polystyrene with a modulus of about 2.1 Gpa. The slot member 441 is reinforced at the slot longitudinal ends 45 with a layer of 0.101 mm thick cold-rolled Type 302 steel, manufactured by Precision Brand, Downers Grove, Ill. The grip portion 69 of the slot member 441 was made from 1 layer of 67 grams per square meter (gsm) (1.8 oz./square yard) nonwoven type #R1159, supplied by BBA of Simpsonville, S.C. Other dimensions include a slot member length L of about 88 mm, a slot length S of about 78 mm, a slot member width W of about 26 mm, and a slot width SW of about 4 mm. The inboard portion 64 is about 5 mm in width in the x-direction. The slot outboard portion 66, not including the grip portion 69 is about 5 mm in width in the x-direction. The slot member 421 is covered on a top surface 448 and a bottom surface 449 with a 30 gsm spunbond nonwoven fabric from BBA, style #088 MLPO 09U. All layers of material in the slot member 441 are adhered to each other with double-sided tape.

The tab member 421 shown in FIGS. 27A and 27B includes a tab load bearing portion 76 comprising a central reinforcing bar about 0.762 mm thick (z-direction) made of high impact polystyrene with a modulus of about 2.1 Gpa. The reinforcing bar is about 10 mm wide in the x-direction and 60 mm long in the y-direction. The tab member 421 also includes a tab stiffening engagement portion 32 that overlaps the tab load bearing portion 76 and extends into the tab grip portion 68. The tab stiffening engagement portion 32 is made of about 0.25 mm polyethylene with a modulus of about 0.65 Gpa and extends about 75 mm in the y-direction and 9.5 mm in the x-direction. The tab member length T is about 75 mm. The tab member width 761 is about 26 mm. The tab member 421 also has an end radius R of about 9.5 mm and a distal width DW of about 9.5 mm. The tab member 421 is covered on a top surface 428 and a bottom surface 429 with a 30 gsm spunbond nonwoven fabric from BBA, style #088 MLPO 09U. All layers of material in the tab member 421 are adhered to each other with double-sided tape.

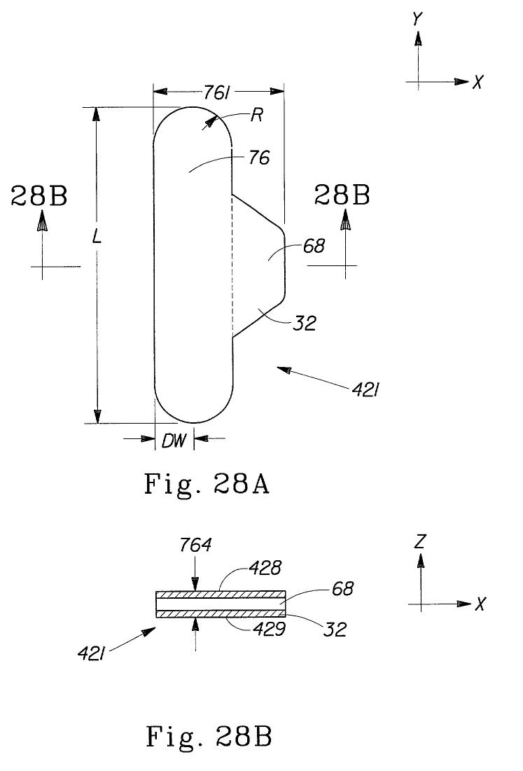

Example 7 is a preferred tab and slot fastening device. The slot member 441 used in example 7 was the same design as used in example 6 and disclosed above. The tab member 421 used in example 7 is shown in FIG. 28A and FIG. 28B. The tab member 421 includes a combined tab load bearing portion 76 and a tab stiffening engagement portion 32. The combined tab load bearing portion 76 and a tab stiffening engagement portion 32 uses the same material to overlap both the tab load bearing portion 76 and the tab grip portion 68. The combined tab load bearing portion 76 and tab stiffening engagement portion 32 is made of about 0.25 mm polyethylene with a modulus of about 0.65 GPa. The tab member length T is about 75 mm. The tab member width 761 is about 26 mm. The tab member 421 also has an end radius R of about 9.5 mm and a distal width DW of about 9.5 mm. The tab member 421 is covered on a top surface 428 and a bottom surface 429 with a 30 gsm spunbond nonwoven fabric from BBA, style #088 MLPO 09U. All layers of material in the tab member 421 are adhered to each other with double-sided tape. The present invention may result in a range of highly flexible tab & slot fastening devices with excellent load bearing capability as demonstrated in examples 6 and 7.

Example 8 is a tab and slot fastening device made of steel. This results in a less desirable stiff fastening device. The slot member 441 of example 8 is similar to that of FIG. 26A and FIG. 26B except that the slot is one piece of 0.889 mm thick (z-direction) stainless steel with no nonwoven covering. Other dimensions include a slot member length L of about 73 mm, a slot length S of about 63 mm, a slot member width W of about 24 mm, and a slot width SW of about 4 mm. The inboard portion 64 is about 5 mm in width in the x-direction. The slot outboard portion 66, not including the grip portion 69 is about 5 mm in width in the x-direction.

The tab member 421 used in example 9 is similar to that shown in FIGS. 28A and 28B except that the tab is one piece of 0.889 mm thick (z-direction) stainless steel with no nonwoven covering. The tab member has a laterally overhanging (x-direction) tab retaining element.

The tab load bearing portion 76 and a tab stiffening engagement portion 32 in this example are the same material (steel). The tab load bearing portion 76 and the tab stiffening engagement portion 32 extend into the tab grip portion 68. The tab member length T is about 60 mm. The tab member width 761 is about 26 mm. The tab member 421 also has an end radius R of about 9.5 mm and a distal width DW of about 9.5 mm.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An in plane engagement fastening device comprising:
a first fastening member, and
a second fastening member, wherein
when the first fastening member and the second fastening member are fastened, the fastening device has a body conformity greater than about 200 percent (%) deflection per kilogram force (kgf).

2. The fastening device of claim 1 wherein the fastening device has a relative deformation of less than about 25% per kgf.

3. The fastening device of claim 1 wherein the fastening device has a body conformity of greater than about 500% per kgf.

4. The fastening device of claim 1 wherein the first fastening member is a tab member, the tab member having a tab load bearing portion with a tab end width and a tab central width, a tab end width to tab central width ratio being greater than about 1.

5. The fastening device of claim I wherein the first fastening device includes a tab member, the tab member includes a tab grip portion with a basis weight, the tab load bearing portion also having a basis weight, a grip portion basis weight to load bearing portion basis weight ratio being less than about 1.

6. The fastening device of claim 1 wherein the first fastening member is a tab member, the tab member having a laterally overhanging tab member.

7. The fastening device of claim 1 wherein the first fastening member is a tab member, the tab member having a multi-plane hinge.

8. The fastening device of claim 1 wherein the first fastening member is a tab member, the tab member having a single plane hinge.

9. The fastening device of claim 1 wherein the second fastening member is a slot member, the slot member having two longitudinal ends with a slot longitudinal end basis weight, and a slot central region with a slot central region basis weight, the slot longitudinal end basis weight to the slot central region basis weight ratio being greater than about 1.

10. The fastening device of claim 1 wherein the second fastening member is a slot member, the slot member having a slot load bearing portion with a slot load bearing portion longitudinal end width and a slot load bearing portion central region width, and a slot load bearing portion longitudinal end width to slot load bearing portion central width ratio greater than about 1.

11. The fastening device of claim 1 wherein the fastening device has a shear load capability in at least one x-direction and at least one y-direction greater than about 50 grams when the first fastening member is fastened with the second fastening member.

12. The fastening device of claim 1 wherein the fastening device is joined to an article having a first region and a second region, such that the first fastening member is joined to the first region and the second fastening member is joined to the second region.

13. The fastening device of claim 12 wherein the first region is a first waist region, the second region is a second waist region, and the fastening device is configured to connect the first waist region with the second waist region to form a waist hoop having an extensibility of at least about 20% under a load of less than about 2000 grams.

14. The fastening device of claim 12 wherein the article is selected from the group consisting of diaper, sanitary napkin, bib, body wrap and article of clothing.

15. The article of claim 1 wherein the first fastening member is selected from the group consisting of slot members, sockets and receptacles; and the second fastening member is selected from the group consisting of tab members, rods, and a projectiles.

16. An article having a first region, a second region opposed to the first region, the article comprising:
a fastening device for joining at least a portion of the first region with at least a portion of the second region, the fastening device including:
a first fastening member, and
a second fastening member, wherein
when the first and second fastening member are engaged, the fastening device has a body conformity greater than about 200% deflection per kgf load.

17. The article of claim 27 wherein the fastening device has a relative deformation of less than about 25% per kgf of load.

18. The article of claim 28 wherein the article is a diaper, catamenial, bib, body wrap or article of clothing.

19. A fastening device comprising:
a tab member including a single plane hinge, and
a slot member.

20. The fastening device of claim 19, wherein when the tab member and the slot member are fastened, the fastening device has a body conformity greater than about 200% deflection per kgf.

21. The fastening device of claim 20 wherein the fastening device has a relative deformation of less than about 25% per kgf.
